SEQUENCE LISTING (4 sequences)

SEQ ID NO: 1 - DNA, 19 bp, synthetic construct
  source 1..19: mol_type = other DNA
actcggtcattctgctcaa

SEQ ID NO: 2 - DNA, 18 bp, synthetic construct
  source 1..18: mol_type = other DNA
ttccagacgcagcgcagt

SEQ ID NO: 3 - DNA, 20 bp, synthetic construct
  source 1..20: mol_type = other DNA
aaaggaagccgcactcagtt

SEQ ID NO: 4 - DNA, 19 bp, synthetic construct
  source 1..19: mol_type = other DNA
tttccagggaagaggggac

(19) United States
(12) Patent Application Publication
(10) Pub. No.: US 2026/0009079 A1
(43) Pub. Date: Jan. 8, 2026

(54) BIOMARKERS OF PREGNANCY LOSS

(71) Applicant: The University of Warwick, Coventry (GB)

(72) Inventors: Jan Brosens, Coventry (GB); Joanne Muter, Coventry (GB); Pavle Vrljicak, Coventry (GB); Emma Lucas, Coventry (GB)

(73) Assignee: The University of Warwick, Coventry (GB)

(21) Appl. No.: 19/121,273

(22) PCT Filed: Nov. 3, 2023
(86) PCT No.: PCT/GB2023/052882

§ 371 Date: Apr. 15, 2025

(30) Foreign Application Priority Data
Nov. 4, 2022 (GB) 2216420.6

(51) Int. Cl. C12Q 1/6883; A61K 31/4985; A61P 15/06

(52) U.S. Cl. CPC C12Q 1/6883; A61K 31/4985; A61P 15/06; C12Q 2600/158

(57) ABSTRACT

The invention relates to methods for assessing the risk of pregnancy loss or embryo implantation failure, and also for monitoring or evaluating the effect of a treatment to reduce the risk of pregnancy loss or embryo implantation failure using specific biomarkers. The invention also relates to the use of these biomarkers in methods of diagnosing a reproductive disorder in an individual, and also to methods of treating a reproductive disorder. In addition, the biomarkers can further be used in methods of selecting patients for treatment to reduce risk of embryo implantation failure or miscarriage. The invention also relates to kits for use in any of the methods described herein.

### FIELD OF THE INVENTION

The invention relates to methods for assessing the risk of pregnancy loss or embryo implantation failure, and also for monitoring or evaluating the effect of a treatment to reduce the risk of pregnancy loss or embryo implantation failure using specific biomarkers. The invention also relates to the use of these biomarkers in methods of diagnosing a reproductive disorder in an individual, and also to methods of treating a reproductive disorder. In addition, the biomarkers can further be used in methods of selecting patients for treatment to reduce risk of embryo implantation failure or miscarriage. The invention also relates to kits for use in any of the methods described herein.

### BACKGROUND TO THE INVENTION

Approximately 15% of clinical pregnancies result in pregnancy loss¹, most often during the first trimester. Fetal chromosomal abnormalities account for 50-60% of miscarriages^{2,3}, and the incidence of aneuploid pregnancies is increasing in developed countries, paralleling the demographic shift towards older maternal age². Aneuploid pregnancy loss is less prevalent in recurrent pregnancy loss (RPL)⁴⁻⁸, defined as two or more losses^{9,10}. With each additional pregnancy loss, the frequency of a euploid loss increases whereas the likelihood of a successful pregnancy decreases⁴. While these observations indicate that maternal factors drive higher-order pregnancy losses, few interventions improve live-birth rates in RPL⁹, reflecting that in many cases the underlying mechanism is unknown.

Mounting evidence from animal studies suggests that dysregulated interactions between the conceptus and endometrium at implantation cause ripple effects that ultimately result in pregnancy failure¹¹⁻¹³. Implantation requires intense remodeling of the endometrial stroma, driven by the postovulatory progesterone surge and rising intracellular cyclic adenosine monophosphate levels¹⁴. This process, termed decidualization, is initiated during the midluteal phase of each cycle and involves differentiation and polarization of endometrial stromal cells (EnSC) into stress-resistant and stressed/senescent decidual subpopulations¹⁵. In parallel, uterine natural killer (uNK) cells accumulate in the stroma and, in response to IL-15 activation, target stressed decidual cells for elimination. Upon implantation, embryo-derived chorionic gonadotrophin rescues the corpus luteum and sustained progesterone signalling promotes the formation of a tightly connected, immune-privileged decidual matrix around the conceptus¹⁶. Thus, a critical challenge at implantation is to simultaneously avoid imminent endometrial breakdown while transforming the cycling endometrium into a semi-permanent tissue, the decidua, maintained throughout pregnancy.

The balance between decidual subpopulations is controlled by extra-uterine cells, foremost natural killer (NK) cells and bone marrow-derived mesenchymal stem cells (MSCs) (FIG. 1). Put simply, balancing decidual subpopulations at implantation from cycle to cycle is controlled by the influx of MSCs, enabling expansion the pool of decidual cells in early pregnancy, and uterine NK cells, which target and eliminate stressed/senescent cells. Recurrent pregnancy loss is associated with both MSC and uNK cell deficiency^{15, 18-19} (Brighton et al., 2017, Lucas et al., 2016, Lucas et al., 2020). Importantly, the level of depletion correlates with the number of previous miscarriages, and by extension, the risk of recurrence^{18,20} (Lucas et al., 2016, Tewary et al., 2020).

Pre-pregnancy screening and intervention may reduce the burden of pregnancy loss. However, currently there are no diagnostic tests to (i) assess the role of endometrial dysfunction in recurrent pregnancy loss, (ii) evaluate the efficacy or therapeutic interventions prior to pregnancy, and (iii) inform patients and their doctors of the risk of future losses of chromosomally normal pregnancies. The Implantation Clinic at the Biomedical Research Unit in Reproductive Health (UHCW) currently provides a test based on quantification of uterine NK cells in midluteal endometrial biopsies using immunohistochemistry (CD56-staining) and image analysis. Because of the intrinsic variability in endometrial uNK cell levels, the test is performed on biopsies obtained in two menstrual cycles. Patients are either self- or GP-referred. The diagnostic pathway is complicated, labour intensive, and dependent on experience staff. Consequently, the results of a test are only available after 5-6 weeks. Another important shortcoming of the current uNK cell test is that it relies on an indirect measure of the endometrial ‘state’.

However, a major challenge in clinical management of recurrent pregnancy loss is differentiation between embryonic and maternal causes of pregnancy failure. Two independent risk factors, maternal age and the number of previous pregnancy losses, have disproportionate effects on pregnancy loss rates (Magnus et al., 2019). The age-specific risk of pregnancy loss is accounted for by the increased incidence of meiotic errors in oocytes, driving the rapid increase in aneuploid embryos after the age of 35 years. On the other hand, the recurrence risk of pregnancy loss increases stepwise with approximately 10% with each additional pregnancy loss independently of maternal age (Magnus et al., 2019). Therefore, an essential criterium for any clinical test that purports to identify maternal factors that are causal to pregnancy loss is that the frequency of a positive test result must increase with each additional pregnancy loss independently of maternal age. None of the pregnancy loss tests currently in clinical practice have been shown to meet this fundamental criterium for causality.

A lack of decidual cells and excess of stressed/senescent stromal cells presents the embryo with an endometrial environment that is easy to invade, devoid of biosensing properties, and prone to breakdown. At a functional level, this pathological endometrial state has been referred to as ‘implantation checkpoint failure’, meaning that the endometrium can neither select against low-fitness embryos nor adequately support high-quality embryos. Both scenarios lead to clinical pregnancy loss^{15, 22-24} (Brosens et al., 2014,Ewington et al., 2019, Brighton et al., 2017, Brosens et al., 2022).

Recent breakthroughs in single-cell RNA sequencing (scRNA-seq) led to the discovery of novel biomarkers of specific endometrial stromal and epithelial subpopulations during the peri-implantation window. The inventors have previously found (as described in WO 2021/032973) that aberrant levels of markers of decidual cells and/or senescent decidual cells were associated with reproductive defects such as miscarriage, in particular SCARA5 and DIO2.

### SUMMARY OF THE INVENTION

The discovery of specific biomarkers of decidual and decidual senescent cells enables assessment of the endometrial ‘state’ during the midluteal implantation window before pregnancy and identify women at risk of subsequent pregnancy loss caused by impaired endometrial function. The present inventors have surprisingly identified that PLA2G2A is a biomarker of pregnancy loss, in particular recurrent pregnancy loss. The biomarker of the present invention is also advantageous when compared to biomarkers described previously, for example SCARA5, as the greater dynamic range in expression of PLA2G2A (as illustrated in FIG. 2B) enhances sensitivity The development of a clinical test based on the biomarkers described herein, including detection of PLA2G2A, can be used in the management of women with a history of one or more prior pregnancy losses. The test can also be used to evaluate the efficacy of pre-pregnancy interventions, including drug treatments, aimed at mitigating the risk of pregnancy loss and increasing live birth rates.

The test of the present invention is designed specifically to assess an endometrial cause of pregnancy loss prior to pregnancy and aid/direct therapeutic interventions. Due to its simplicity and ease, it has the potential to become a routine, quick and relatively inexpensive test for any women suffering pregnancy loss, even if for the first time. It is a simpler and less expensive alternative to the currently available commercial test aimed at determining the ‘window of implantation’ in IVF patients (described in EP2 333 107 B1). The test of the present invention, importantly, shows an increase in the frequency of positive results with each additional pregnancy loss independently of maternal age, and therefore can be said to identify maternal factors that are causal to miscarriage.

Thus, the present inventors have surprisingly identified that PLA2G2A can be used as a biomarker alone or in combination in methods for assessing the risk of pregnancy loss or embryo implantation failure, and also for monitoring or evaluating the effect of a treatment to reduce the risk of pregnancy loss or embryo implantation failure. The biomarkers described herein can also be used in methods of diagnosing a reproductive disorder in an individual, and also to assist methods of treating a reproductive disorder. In addition, these biomarkers can further be used in methods of selecting patients for treatment to reduce risk of embryo implantation failure or miscarriage, methods of stratifying patients. The biomarkers may be detected using kits as described herein.

The present invention thus provides a method for assessing the risk of pregnancy loss or embryo implantation failure in an individual, wherein the method comprises detecting and/or quantifying the amount of at least one marker gene for decidual cells and at least one marker gene for decidual senescent cells in a biological sample obtained from the individual, and thereby assessing the risk, wherein the at least one marker gene for decidual cells comprises PLA2G2A.

The present invention also provides a method of monitoring or evaluating the effect of a treatment to reduce the risk of pregnancy loss or embryo implantation failure in an individual, wherein the method comprises detecting and/or quantifying the amount of at least one marker gene for decidual cells and at least one marker gene for decidual senescent cells in a biological sample obtained from the individual, and thereby monitoring or evaluating the effect of the treatment, wherein the at least one marker gene for decidual cells comprises PLA2G2A.

The present invention further provides a method of diagnosing a reproductive disorder in an individual, wherein the method comprises detecting and/or quantifying the amount of at least one marker gene for decidual cells and at least one marker gene for decidual senescent cells in a biological sample obtained from the individual, thereby diagnosing the disorder, wherein the at least one marker gene for decidual cells comprises PLA2G2A.

The present invention also provides a method of treating a reproductive disorder in an individual, or of preventing pregnancy loss or embryo implantation failure in an individual, the method comprising diagnosing the reproductive disorder or assessing the risk of pregnancy loss or embryo implantation failure as described herein, and administering an agent or carrying out a treatment regimen effective to treat the reproductive disorder or prevent pregnancy loss or embryo implantation failure in the individual who is positively diagnosed or assessed as being at risk.

The present invention also provides a method of selecting patients for treatment to reduce risk of embryo implantation failure or miscarriage, wherein the method comprises detecting and/or quantifying the amount of at least one marker gene for decidual cells and at least one marker gene for decidual senescent cells in a biological sample obtained from an individual, and selecting the patients for treatment to reduce the risk of pregnancy loss or embryo implantation failure based on the level of the marker genes,, wherein the at least one marker gene for decidual cells comprises PLA2G2A.

The present invention also provides a test kit suitable for use in a method described herein, wherein the test kit comprises means for detecting or quantifying at least one marker gene for decidual cells and at least one marker gene for decidual senescent cells at a nucleic acid or protein level, and optionally means for detecting and/or quantifying the level of UNK cells or the level of at least one marker gene for uNK cells in the individual.

The invention further provides a method of assessing readiness for conception or successful embryo implantation in an individual comprising detecting and/or quantifying the amount of at least one marker gene for decidual cells and at least one marker gene for decidual senescent cells in a biological sample obtained from the individual, and thereby assessing readiness for conception or successful embryo implantation, wherein the at least one marker gene for decidual cells comprises PLA2G2A.

The present invention provides a particularly advantageous endometrial test for recurrent pregnancy loss patients. The test is suitable for any woman planning pregnancy.

### BRIEF DESCRIPTION OF THE FIGURES

FIG. 1: Mechanisms influencing the decidual pathway

Transition of the cycling endometrium into the decidua of pregnancy requires cooperation between decidual cells and uterine natural killer (uNK) cells to eliminate senescent decidual cells. In parallel bone marrow-derived mesenchymal stem cells (MSCs) are recruited to ensure rapid tissue expansion in pregnancy. Lack of MSCs and/or uNK cells drive a pro-senescent decidual response that renders the uteroplacental interface vulnerable to tissue breakdown in pregnancy, thereby causing miscarriage. SASP, senescence-associated secretory phenotype; IL-15, interleukin 15.

FIG. 2: Expression of decidual cell and decidual senescent cell markers in the endometrium

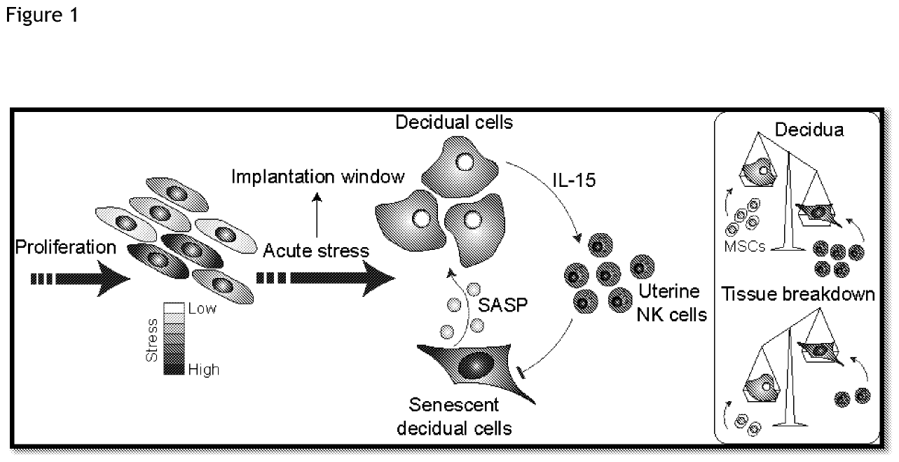

(A) SCARA5 and DIO2 percentiles of 6 pairs of endometrial biopsies (n=12) subjected to RNA-seq. Open and closed circles designate “A” and “B” biopsies, respectively. Paired biopsies indicated by dashed lines. Samples in the upper left quadrant (SCARA5 mRNA levels<30 percentile and DIO2>70 percentile) were deemed ‘abnormal’, that is, harbouring an excess of stressed/senescent cells and lacking decidual cells. (B) Volcano plot indicating differential gene expression between endometrial samples deemed ‘normal’ and ‘abnormal’ revealed that high DIO2 expression is associated with low PLA2G2A expression, and vice versus. Bonferroni correction at 0.05 indicated by dashed line. (C) Violin plots showing expression of DIO2 and PLA2G2A mRNA expression in endothelial, epithelial, immune and stromal cells from single cell in vivo in luteal phase endometrial biopsies. (D) DIO2 and PLA2G2A mRNA expression in proliferative and early-, mid-, and late-luteal phase endometrium. Each bar represents an individual biopsy. The data were retrieved from microarray data deposited in the Gene Expression Omnibus (GEO Profiles ID: GDS2052). (E) DIO2 and PLA2G2A mRNA levels quantified by RT-qPCR analysis in 822 endometrial biopsies obtained between LH+6 and LH+11. Percentile graphs showing the distribution of gene expression across the peri-implantation window were generated based on normalized expression values using R software. The median number of samples for each day was 153 (range: 51-202).

FIG. 3: Spatial organisation of PLA2G2A- and DIO2-expressing cells in the endometrium

Spot-plots depicting spatial log-transformed normalized expression of DIO2 (upper panel) and PLA2G2A (lower panel) in a formalin-fixed, paraffin-embedded endometrial tissue section (LH+8). Original magnification 10x.

FIG. 4: Association of PLA2G2A/DIO2 ratios with the number of previous miscarriages

(A) Distribution of PLA2G2A/DIO2 percentiles in 854 LH-timed endometrial biopsies in patients with a history of 0 to 18 miscarriages. Different letters above columns indicate significance at P<0.05 following one-way ANOVA with Tukey's multiple comparisons test. (B) Frequency of a endometrial biopsy with a PLA2G2A/DIO2 ratio in the lower quartile as a function of the number of previous miscarriages. (C) Frequency of endometrial samples with PLA2G2A/DIO2 ratios <15^{th} percentile as a function of the number of previous losses. Total n numbers for each column are indicated. Different letters above columns indicate significance at P<0.05 following Chi-square test with repeated measures. (D) Comparison of age (upper panel), BMI (middle panel) and day of biopsy post luteinizing hormone surge (LH+, lower panel) between samples with a PLA2G2A/DIO2 ratio >15th percentile (designated ‘normal’) and <15^{th} centile (‘abnormal’). Students 1-test; ** denotes P<0.01; n.s.: non-significant (P>0.05).

FIG. 5: Use of PLA2G2A/DIO2 ratios to predict the risk of future pregnancy loss

(A) Analysis of PLA2G2A/DIO2 ratios in endometrial biopsies obtained prior to a pregnancy that resulted in live birth or miscarriage. Left panel show the relative proportion of live birth versus pregnancy loss in pre-pregnancy endometrial biopsies with PLA2G2A/DIO2 ratios below the indicated percentiles. Right panel show the relative proportion of live birth versus pregnancy loss in pre-pregnancy endometrial biopsies with PLA2G2A/DIO2 ratios above the indicated percentiles. Chi-square test, * denotes P<0.05. n.s.: non-significant (P>0.05). (B) Age, body mass index (BMI) and endometrial uNK cell percentiles prior to a subsequent pregnancy that resulted in live birth or miscarriage. Students 1-test; n.s.: non-significant (P>0.05)

FIG. 6: Effect of oral sitagliptin on endometrial PLA2G2A/DIO2 ratios

DIO2 and PLA2G2A transcripts were measured by RT-qPCR in paired baseline and second endometrial biopsies obtained from participants in the SIMPLANT trial (EudraCT Number 2016-001120-54) in the placebo (n=16) and sitagliptin (n=15) groups. The PLA2G2A/DIO2 ratios (bottom panel) were calculated. The data were analyzed using Wilcoxon Matched Pairs Signed Ranks test with Sidak correction

### DETAILED DESCRIPTION OF THE INVENTION

It is to be understood that different applications of the disclosed methods may be tailored to the specific needs in the art. It is also to be understood that the terminology used herein is for the purpose of describing particular embodiments of the invention only, and is not intended to be limiting. All publications, patents and patent applications cited herein, whether supra or infra, are hereby incorporated by reference in their entirety.

As used in this specification and the appended claims, the singular forms “a”, “an”, and “the” include plural referents unless the content clearly dictates otherwise. Thus, for example, reference to “a cell” includes “cells”, and the like.

### Method for Assessing the Risk of Pregnancy Loss or Embryo Implantation

The invention provides a method for assessing the risk (or likelihood or probability) of pregnancy loss or embryo implantation failure in an individual, wherein the method comprises detecting and/or quantifying the amount of at least one marker gene for decidual cells and at least one marker gene for decidual senescent cells in a biological sample obtained from the individual, and thereby assessing the risk, wherein the at least one marker for decidual cells comprises PLA2G2A. In the above method and all other methods and aspects of the invention, PLA2G2A is detected as a decidual marker gene. In other words, at least one of the decidual marker genes detected is PLA2G2A. The decidual marker genes used for detection thus comprise at least PLA2G2A. The methods may further comprise the detection of additional decidual marker genes.

Any further marker genes for decidual cells and at least one marker gene for decidual senescent cells may be selected from any such marker genes. Thus, the marker genes may be any genes indicative of the levels of decidual cells and decidual senescent cells in the sample, such as an endometrial sample. For example, a marker gene for decidual cells may be any marker whose decrease is indicative of a decreased level of decidual cells, and a marker gene for decidual senescent cells may be any marker whose increase is indicative of an increased level of decidual senescent cells. For example, further marker genes for decidual cells may be selected from SCARA5, FTL, GLRX and/or IL1RL1. The at least one marker gene for decidual senescent cells may preferably comprise DIO2. The at least one marker gene for decidual senescent cells may be selected from DIO2, CLU and IGFBP1.

The level of the marker genes is typically compared with a control sample or reference sample or level. Any suitable control sample or reference sample or level may be used. A control sample or reference sample or level may represent a normal or healthy sample/level, for example obtained or determined from an individual or multiple individuals not having any reproductive disorder, or not having had pregnancy loss or embryo implantation failure. The individual(s) may have had one or more successful pregnancies. The control or reference sample or level may also be obtained or determined from an individual or multiple individuals who have responded positively to treatment to reduce the risk of pregnancy loss or embryo implantation failure. Alternatively, a control sample or reference sample or level may represent a sample/level from an individual or multiple individuals having a reproductive disorder, or who have had one or more miscarriages or embryo implantation failures, i.e. a positive control or reference sample or level. In the case of control or reference samples or levels from multiple individuals, an average value may be obtained or the results may be pooled to generate a more accurate reference range. The level determined in the test sample is preferably compared to a control or reference sample or level that is obtained on or around the same day of the menstrual cycle as the test sample. Other suitable control or reference samples or levels can readily be identified by the person skilled in the art.

A decreasing or decreased level of a marker gene for decidual cells e.g. PLA2G2A in a sample, as compared with a reference sample or level, may indicate that the individual is at risk of pregnancy loss or embryo implantation failure. Alternatively, an increasing or increased level of a marker gene for decidual senescent cells e.g. DIO2, as compared with a reference sample or level, may indicate that the individual is at risk of pregnancy loss or embryo implantation failure. The level of marker gene can be determined using any methods described herein and that are known to the skilled person. In one instance, a decreasing or decreased level of a marker gene for decidual cells e.g. PLA2G2A, as compared with a reference sample or level, and an increasing or increased level of a marker gene for decidual senescent cells e.g. DIO2, as compared with a reference sample or level, together indicate that the individual is at risk of pregnancy loss or embryo implantation failure.

In one instance, the method for assessing the risk of pregnancy loss or embryo implantation failure in an individual further comprises detecting and/or quantifying the level of uterine natural killer (uNK) cells in the sample, for example based on the level of at least one marker gene for uNK cells in the sample.

A decreasing or decreased level of the uNK cells or uNK cell gene markers in a sample, as compared with a reference sample or level, may indicate that the individual is at risk of pregnancy loss or embryo implantation failure.

The method of the invention may also further comprise detecting and/or quantifying genes that allow identification of the day in the menstrual cycle as described below.

Further risk indicia, which may be used to assess the risk of pregnancy loss or embryo implantation failure, include maternal body mass index (BMI), maternal age, number of previous pregnancy losses or embryo implantation failures, familial and intergenerational factors, history of infertility, placental abnormalities, cervical and uterine anomalies, smoking, alcohol consumption, etc. Additional risk indicia are known to those of skill in the art.

For instance, there may be an increased risk of pregnancy loss or embryo implantation failure if the maternal BMI is too low (e.g. <18.5) or too high (e.g. >25), if the maternal age is 35 and above, if there has been a history of recurrent pregnancy loss (e.g. when a woman has had 2 or more pregnancy losses before the pregnancies reached 20 weeks).

### Samples

As used herein, the term “biological sample” or “sample” refers to any sample that is taken from an individual. Suitable samples in the context of the methods of the present invention include, for example, endometrial tissue, endometrial secretions, cells obtained from the endometrium, or an endometrial biopsy sample.

A sample obtained from the endometrium may be collected by any method known in the art, including through an endometrial biopsy or endometrial sampling. The technique involves removing a piece of tissue from the inner lining of the uterus (endometrium). The sample may also be obtained using a dilation and curettage procedure.

The sample may be or may have been processed prior to use, for example by dilution, centrifugation or extraction of DNA, RNA or protein. The sample may be a freshly obtained sample or may be or have been stored or preserved, e.g. by freezing, prior to use.

The sample may be taken during the luteal phase of the menstrual cycle. The luteal phase begins with the formation of the corpus luteum, with progesterone being significantly higher than in other phases of the menstrual cycle. The sample may be taken during the mid-luteal phase of the menstrual cycle. The sample is thus typically taken post-ovulation. The sample is typically taken during the embryo implantation window (also known as the receptivity window), of the menstrual cycle, in which the endometrium is receptive to implantation of an embyro. The embryo implantation window may be determined by any means, and may for example be calculated based on an ovulation test. An ovulation test may be based on hormone level, such as luteinizing hormone (LH) level (for example LH level in urine) or oestrogen level (for example based on salivary ferning). Alternatively, the level of one or more markers indicative of receptivity to embryo implantation may be determined. The sample may be taken between about 5 and about 11 days after an increase or surge (such as a 2-5 fold increase or surge) in the level of LH s, i.e. LH+5 to LH+11. LH is produced by the pituitary gland and is generally secreted at very low levels throughout the menstrual cycle, with the ovulatory phase of the menstrual cycle however beginning with a surge in LH.

The sample type and timing of sampling described above is applicable to any of the methods of the present invention detecting marker levels.

### Individual

The individual referred to in any of the methods of the invention may be a human or a non-human menstruating mammal. The methods described herein may thus be applied in a veterinary context. The subject is preferably a human female.

The individual may be suffering or have suffered from infertility or embryo implantation failure. For instance, the individual may suffer from or have suffered from embryo implantation failure following in vitro fertilisation treatment. The individual may have suffered from at least one previous pregnancy loss or multiple pregnancy losses, and/or at least one embryo implantation failure or multiple embryo implantation failures. The individual may suffer from recurrent pregnancy loss (RPL).

The individual may already be considered to be at risk of pregnancy loss or embryo implantation failure. The individual may be considered at risk of pregnancy loss or embryo implantation failure due to the presence of one or more of the risk indicia, including low or high body mass index (BMI), maternal age, number of previous pregnancy losses or embryo implantation failures, familial and intergenerational factors, history of infertility, placental abnormalities, cervical and uterine anomalies etc. Additional risk indicia for pregnancy loss or embryo implantation failure are known to those of skill in the art.

### Pregnancy Loss

Pregnancy loss refers to the failure of an embryo to result in a baby, which may result from unsuccessful embryo implantation. Pregnancy loss as assessed according to the biomarkers of the present invention is typically loss in the first trimester, particularly in the first 20 to 23 weeks of gestation, which may also be referred to as “miscarriage”. Typical symptoms of a miscarriage comprise vaginal bleeding with or without pain, and also cramping and pain in the lower abdomen. When at least two or more miscarriages occur, an individual may be diagnosed as suffering from recurrent pregnancy loss (RPL) or infertility. However, RPL may not be a binary state and there may be an increased risk of further pregnancy loss with any previous loss. The invention allows for the detection of any predisposition to RPL irrespective of the number of previous losses.

A majority of miscarriages are thought to be caused by chromosomal abnormalities or errors in the embryo, such as aneuploidy, e.g. autosomal trisomy, monosomy X, triploidy, tetraploidy etc. Other miscarriages are not due to chromosomal abnormalities or errors in an embryo.

In a method of the present invention, the risk of pregnancy loss/miscarriage may be assessed. The method of the invention is preferably used for determining the risk of euploid miscarriage, and where the cause of pregnancy loss is not due to a chromosomal abnormality or error in an embryo. The method of the invention is preferably used for determining the risk of recurrent pregnancy loss/miscarriage.

### Embryo Implantation Failure

After fertilisation, a fertilised egg (or zygote) begins to divide by mitosis to produce an embryo. The process of the embryo attaching to the lining of the uterus, i.e. the endometrium, is known as implantation. “Embryo implantation failure” or “implantation failure” in accordance with the invention refers to failure of an embryo to be implanted into the endometrium. Implantation failure may occur either where patients are trying to conceive naturally, without any fertility treatment or after undergoing assisted reproductive technology e.g. in vitro fertilisation (IVF).

Some cases of implantation failure are thought to be caused by chromosomal abnormalities in the embryo, such as aneuploidy, e.g. autosomal trisomy, monosomy X, triploidy, tetraploidy etc. Other cases of embryo implantation failure are not due to chromosomal abnormalities or errors in an embryo. Some cases of implantation failure related to IVF are caused by poor embryo quality, the age of the eggs, a lack of response to IVF medication or other lifestyle factors (e.g. smoking).

According to the present invention, the risk of any case of embryo implantation failure may be assessed in any setting. The method of assessing risk of embryo implantation failure of the invention may thus be used for determining risk of implantation failure following natural conception, or the risk of implantation failure following assisted reproduction in an individual, for example following in vitro fertilisation. The method is preferably used for determining the risk of embryo implantation failure that is not due to a chromosomal abnormality or error in an embryo.

### Decidual Cells and Decidual Senescent Cells and Marker Genes

In accordance with the method of assessing risk of pregnancy loss or implantation failure of the present invention, at least one marker gene for decidual cells and at least one marker gene for decidual senescent cells are detected and/or quantified, wherein the at least one marker gene for decidual cells comprises PLA2G2A.

When exiting the cell cycle, a cell either differentiates into a specialised cell or becomes senescent. Cellular senescence is a defined cellular state, which can be acute or chronic. A key characteristic of senescent cells (SNCs) is that they are in a state of permanent cell-cycle arrest, typically initiated and maintained by the p53-p21-retinoblastoma (RB) and p16-RB tumour suppressor pathways. SNCs produce a bioactive “secretome,” referred to as the senescence-associated secretory phenotype (SASP), which can disrupt normal tissue architecture and function through diverse mechanisms, including recruitment of inflammatory immune cells, remodelling of the extracellular matrix, induction of fibrosis, and inhibition of stem cell function⁵⁸.

During decidualization, the cells of the endometrium undergo significant changes in preparation for and during pregnancy. During this process, endometrial stromal cells (EnSC) either become a specialised cell (i.e. decidual cell) or become acutely senescent (i.e. decidual SNC).

The decidual cell detected may be any decidual cell and the decidual senescent cell detected any decidual senescent cell, typically any such cells in an endometrial sample. A decidual or decidual senescent cell is typically derived from an endometrial stromal cell. Decidual cells are stress-resistant and are also described herein as stress-resistant decidual cells.

In accordance with the method of assessing risk of pregnancy loss or implantation failure of the present invention, at least one marker gene for decidual cells and at least one marker gene for decidual senescent cells may be detected, wherein the at least one marker gene for decidual cells comprises PLA2G2A. The at least one marker gene for decidual cells may further comprise one or more of SCARA5, FTL, GLRX and IL1RL1, and the marker genes for decidual senescent cells typically comprise one or more DIO2, CLU and IGFBP1. The method may comprise detecting a decrease in PLA2G2A. Preferably, the at least one marker gene for decidual senescent cells is DIO2. The method may comprise detecting an increase in DIO2. In a preferred embodiment, the at least one marker gene for decidual senescent cells is DIO2, and the method comprises detecting and/or quantifying the amount of both PLA2G2A and DIO2. The method may comprise detecting a decrease in PLA2G2A and an increase in DIO2. Any of the above genes may be detected and/or quantified in the method for assessing the risk of pregnancy loss or embryo implantation failure of the invention, as well as in further methods described below.

Centile or percentile graphs may be employed to compare expression levels of marker genes such as marker genes of decidual and decidual senescent cells (and also levels of other markers as discussed below) in samples obtained at different days in the menstrual cycle. Centile graphs are based on the statistical distribution of the expression levels of a given marker gene on a given day in the menstrual cycle, e.g. following a positive ovulation test. The more samples used to generate the centile graphs, the more accurate the reference range. For instance, centile graphs may be based on at least 10 samples, at least 100 samples, at least 250 samples, at least 500 samples, at least 1000 samples, at least 2000 samples, at least 5000 samples, or more. The relative expression level of a given marker gene (i.e. centile) in a sample of an individual obtained on a given day in the menstrual cycle may be calculated against the reference percentile graph.

In some instances, the determination of the centile for each marker gene allows the cause and clinical presentation of the individual to be determined, such as that for recurrent pregnancy loss. Putative defects, or decidual dyshomeostasis, along the decidual pathway may be determined. In one example, a low level of a decidual cell marker gene e.g. PLA2G2A and a high level of decidual senescent cell marker gene e.g. DIO2, as compared with a reference sample or reference level, is indicative of excessive decidual senescence, and occurs more frequently in recurrent pregnancy loss. In a particular instance, where the ratio of PLA2G2A to DIO2 is below the 50^{th} percentile as compared with a reference sample or reference level, then there is a positive diagnosis. In other instances, where the ratio of PLA2G2A to DIO2 is below the 40^{th} percentile, the 30^{th} percentile, the 20^{th} percentile, or the 10^{th} percentile, as compared with a reference sample or reference level, then there is a positive diagnosis.

In other instances, the invention may comprise determining the relative risk of miscarriage. For example, an individual may be found to be twice as likely to miscarry as to have a live birth. In other aspects, the individual may be found to be three, four or five times as likely to miscarry. In one particular instance, an individual may be found to be twice as likely to miscarry when the ratio of PLA2G2A to DIO2 is below the 15^{th} percentile.

### uNK Cells and uNK Cell Gene Markers

In accordance with the method of assessing risk of pregnancy loss or implantation failure of the invention, in addition to the marker genes for decidual cells and decidual senescent cells, levels of uterine natural killer (uNK) cells may also be detected and/or quantified, typically by detection of one or more marker genes for uNK cells.

Successful transition of the endometrium (from a cycling tissue into a semi-permanent tissue capable of maintaining the placenta throughout pregnancy) is dependent on stress-resistant decidual cells co-opting uterine natural killer (uNK) cells to eliminate their acutely stressed counterparts, i.e. decidual senescent cells, through granule exocytosis. Thus, the balance of diverging decidual populations and uNK cells during the midluteal phase of the menstrual cycle is described herein to likely determine the ability of the endometrium to transition into a pregnancy tissue. Imbalance in decidual subsets is also linked herein to reproductive failure. Thus, determination of the level of uNK cells in combination with detecting levels of markers for decidual and decidual senescent cells provides additional information for assessment of the risk of pregnancy loss or implantation failure and also in relation to diagnosis of reproductive disorders more generally.

The level of uNK cells in the sample may be detected and/or quantified by any means known in the art. uNK cells may be detected and/or quantified using immunohistochemistry and image analysis. Alternatively, uNK cells may be detected based on the level of at least one marker gene for uNK cells in the sample.

In accordance with the methods of the present invention, any uNK cell gene marker may be detected and/or quantified. Marker genes for uNK cells may be selected from NCAM1, KLRB1, KLRC1, GZMA, GZMB, IL2RB and IL2RG. The above genes may be detected and/or quantified in the method for assessing the risk of pregnancy loss or embryo implantation failure described herein, and also in further methods of the invention detecting marker genes as described below. uNK cell deficiency occurs more frequently in recurrent pregnancy loss.

### Determination of the Timing in the Menstrual Cycle

In accordance with the methods of the present invention, a determination of the time (such as the point, stage or day) in the menstrual cycle on which the sample is obtained may be additionally carried out. Any parameter including any known hormone, marker or other parameter (including any hormone or marker described above) that allows timing of the point, stage or day in the cycle may be used. Preferably, the sample is obtained in the embryo implantation window, and the point, stage or day within the embryo implantation window is determined. The determination of the point, stage or day in the menstrual cycle advantageously allows a sample to be compared to a reference sample or level representative of the same point, stage or day, since the hormone levels or marker gene levels change throughout the cycle.

In a particular embodiment, in addition to marker genes for decidual cells, decidual senescent cells and/or uNK cells, marker genes that allow identification of the point, stage or day in the menstrual cycle are thus also detected and/or quantified. Such marker genes are also referred to herein as molecular timing genes, and are typically indicative of timing in the implantation window. Through the analysis of these genes, the accuracy of the detection based on analysis of the marker genes for decidual cells and decidual senescent cells and/or uNK cell or uNK cell marker genes may be improved.

The purpose of molecular timing is two-fold. Because of cycle-dependence of gene marker levels, the interpretation of the levels of the decidual cell gene markers e.g. PLA2G2A, the decidual senescent cell gene markers e.g. DIO2 and/or uNK cell levels or uNK cell gene markers, is advantageously assisted by knowledge of the day in the cycle a biopsy is taken. Practically, this may also be achieved by scheduling the biopsy relative to the pre-ovulatory luteinising hormone (LH) surge as discussed above. The methods of the invention may thus be carried out in an individual by obtaining a sample at a suitable time point subsequent to an LH surge as described above. However, by considering molecular timing based on analysis of marker gene expression, the risk of erroneous timing of the biopsy due to patient error and intrinsic variation between the LH surge and the exact time of ovulation may be reduced.

The window of implantation (also known as the receptivity window) is associated with dramatic changes in gene expression in the glandular epithelium. Thus, the method of the invention may preferably comprise detection of any marker gene having a change in expression (and which is typically selectively expressed) in the glandular epithelium during the implantation window and thus able to report on the point, stage or day in the embryo implantation window. The determination may preferably be based on two or more genes that are selectively expressed in the glands and that exhibit opposing expression profiles as the menstrual cycle progresses. The ratio of two or more such genes may be determined.

Marker genes that enable determination of the molecular timing of the embryo implantation window and may be used according to the invention include any one or more of GPX3, DPP4 (a GPX3-like gene), SLC15A2 and CTNNA2 (a SLC15A2-like gene). Preferably, the genes that allow identification of the timing of the day in the menstrual cycle may comprise, consist of, or consist essentially of, GPX3 and SLC15A2. The ratio of GPX3 and SLC15A2 may thus be determined. Because molecular timing as used in the invention is typically based on genes selectively expressed in epithelial cells such as GPX3 and SC15A2, whereas decidual cell and decidual senescent cell markers e.g. PLA2G2A and DIO2 are selective stromal cell markers, molecular timing may also be used to diagnose asynchrony between the hormonal response in the epithelial and stromal compartment. As described herein, GPX3 and SLC15A2 are regulated in opposing ways as the luteal phase unfolds (i.e. GPX3 is rapidly upregulated whereas SLC15A2 is rapidly downregulated). Consequently, the ratio of these two genes changes profoundly from day to day during the implantation window. The ratio between GPX3 and SLC15A2 rises markedly across LH+5 and LH+11 days of the cycle and thus the ratio between GPX3 and SLC15A2 may be matched to a particular day in the cycle. The particular GPX3/SLC15A2 ratio may be matched to a particular day in the cycle based on a centile/percentile graph plotted from values obtained from pooled reference samples for that particular day in the cycle, for example as obtained by detection of the LH surge (e.g. using home ovulation kits). Preferably, if the GPX3/SLC15A2 ratio in a test sample falls between the 25^{th} to 75^{th} percentile of a reference centile/percentile graph plotted from values obtained from pooled reference samples for a particular day in the cycle, and matches (i.e. is congruent) with the day of the luteal phase as determined by detection of the LH surge (e.g. using a home ovulation kit), then the timing of a biopsy may be considered improved in accuracy, and test results may be reported on the basis of the day in the cycle as determined by detection of the LH surge e.g. using a home ovulation kit. If the GPX3/SLC15A2 ratio in a test sample falls outside of the 25^{th} to 75^{th} percentile of a reference centile/percentile graph plotted from values obtained from pooled reference samples for a particular day in the cycle (i.e. is incongruent), or if the GPX3/SLC15A2 ratio does not match with the day of the luteal phase as determined by detection of the LH surge (e.g. using a home ovulation kit), then the timing of a biopsy may be considered less accurate, and test results may be reported both on the basis of the day in the cycle as determined by detection of the LH surge e.g. using a home ovulation kit, as well as based on molecular timing results.

### Marker Gene Sequences

Particular sequences for marker genes useful in accordance with the present invention, with their database accession/identification number in the NCBI Gene database, Ensembl database and OMIM database are disclosed herein. The gene sequences as disclosed herein include those available with reference to these online sequence databases as of 16 Jun. 2019. Thus, below is a list of marker genes with representative accession numbers (in parentheses: NCBI Gene database, followed by Ensembl database, followed by OMIM database) and alternative gene names (in italics):
Phospholipase A2 Group IIA: PLA2G2A (5320, ENSG00000188257, 172411) MOM1, PLA2, PLA2B, PLA2L, PLA2S, PLAS1, sPLA2
Scavenger Receptor Class A Member 5: SCARA5 (286133, ENSG00000168079, 611306) Tesr, NET33, FLJ23907, MGC45780;
Ferritin Light Chain: FTL (2512, ENSG00000087086, 134790) LFTD, NBIA3, MGC71996;
Glutaredoxin: GLRX (2745, ENSG00000173221, 600443) GRX, GRX1;
Interleukin 1 Receptor Like 1: IL1RL1 (9173, ENSG00000115602, 601203) T1, ST2, DER4, ST2L, ST2V, FIT-1, IL33R;
Iodothyronine Deiodinase 2: DIO2 (1734, ENSG00000211448, 601413) D2, 5DII, SelY, DIOII, TXDI2;
Clusterin: CLU (1191, ENSG00000120885, 185430) CLI, AAG4, APOJ, CLU1, CLU2, KUB1, SGP2, APO-J, SGP-2, SP-40, TRPM2, TRPM-2, NA1/NA2;
Insulin Like Growth Factor Binding Protein 1: IGFBP1 (3484, ENSG00000146678, 146730) AFBP, IBP1, PP12, IGF-BP25, hIGFBP-1;
Glutathione Peroxidase 3: GPX3 (2878, ENSG00000211445, 138321) GPx-P, GSHPx-3, GSHPx-P;
Solute Carrier Family 15 Member 2:
SLC15A2 (6565, ENSG00000163406, 602339) PEPT2;
Dipeptidyl-peptidase IV: DPP4 (1803, ENSG00000197635, 102720) CD26, ADABP, ADCP2, DPPIV, TP 103;
Catenin Alpha 2: CTNNA2 (1496, ENSG00000066032, 114025) CAPR, CTNR, CAP-R, CT114, CDCBM9;
Interleukin 2 Receptor Subunit Beta: IL2RB (3560, ENSG00000100385, 146710) CD122, IL15RB, P70-75;
Interleukin 2 Receptor Subunit Gamma: IL2RG (3561, ENSG00000147168, 308380) P64, CIDX, IMD4, CD132, SCIDX, IL-2RG, SCIDX1;
Neural Cell Adhesion Molecule 1: NCAM1 (4684, ENSG00000149294, 116930) CD56, NCAM,MSK39.

### Detection and/or Quantification of the Amount Level of the Biomarkers

As used herein, the term “marker gene” or “biomarker” refers to a gene, or a fragment of a gene, the change in amount and/or the detection of which can be correlated with a particular physical condition or state. Particular marker genes used in the present invention are correlated with the risk of pregnancy loss or embryo implantation failure, and are also used in the methods described herein. The detection and/or quantification of such marker genes may be achieved by any means and is not limited to detection/quantification of nucleic acids. Marker genes may also be detected via their respective expression products, including the expressed peptides, polypeptides, and proteins, and fragments thereof.

As used herein, the term “amount” or “level” as used herein refers to a quantity of a marker gene or its expression product that is detectable or measurable in a biological sample and/or control or reference sample. The quantity of a marker gene can be, for example, a quantity of nucleic acid or protein. The term can alternatively include combinations thereof. The amount or level of the marker genes may refer to the absolute amount or level of the biomarkers. Alternatively, a change in the relative level or amount of marker(s) may be assessed by comparing the level or amount of the marker genes in a sample from the subject with a control value or reference value. Alternatively, the relative amount or level of the marker genes may in some instances refer to the concentration of the marker genes relative to the total amount or level of marker genes in the sample.

The level of marker genes can be detected and/or quantified by detection of nucleic acid, e.g. RNA. For example, levels of mRNA can be measured by reverse transcription quantitative polymerase chain reaction (RT-PCR followed with qPCR). RT-PCR is used to create a cDNA from the mRNA. The cDNA can be used in a qPCR assay to produce fluorescence as the DNA amplification process progresses. By comparison to a standard curve, qPCR can produce an absolute measurement such as number of copies of mRNA per cell. Northern blots, microarrays, Invader assays, and RT-PCR combined with capillary electrophoresis may be used to measure expression levels of mRNA in a sample.

In some embodiments, nucleic acid amplification methods can be used to detect a polynucleotide biomarker. For example, oligonucleotide primers and probes can be used in amplification and detection methods that use nucleic acid substrates isolated by any of a variety of well-known and established methodologies. Methods for amplifying nucleic acids include, but are not limited to, for example the polymerase chain reaction (PCR) and reverse transcription PCR (RT-PCR), ligase chain reaction (LCR), strand displacement amplification (SDA), thermophilic SDA (tSDA), Taqman-PCR, multiplex Taqman-PCR, Nanostring, targeted sequencing, digital PCR or any suitable method known in the art. In a preferred embodiment, the level of marker genes can be detected and/or quantified by droplet digital qPCR (ddPCR).

The detection and quantification of marker genes in the methods of the invention may also involve the use of an agent wherein the agent specifically detects expression products of the marker genes, e.g. proteins or peptides of interest. The agent could be an antibody or functional equivalent thereof that binds proteins or peptides under analysis (i.e. anti-peptide antibody). These antibodies may be used to perform an immunoassay such as, but not limited to, enzyme linked immunosorbent assay (ELISA), radio-immunoassay, immunoprecipitation, immunohistochemistry, immunofluorescence, protein dot blot, Western blot, turbidimetry, nephelometry, FACS and the like, which are known to the skilled person.

The relative abundances of the marker genes for decidual cells and decidual senescent cells may be expressed as a ratio, e.g. a PLA2G2A/DIO2 ratio. The fold-change in this ratio provides information regarding the respective levels of these marker genes and may be used in the methods described herein. An increase in the level of marker genes for decidual cells, and optionally also a decrease in the level of marker genes for decidual senescent cells, would lead to an increased ratio. A decrease in the level of marker genes for decidual cells, and optionally also an increase in the level of marker genes for decidual senescent cells, would lead to a decreased ratio.

### Method of Monitoring or Evaluating the Effect of a Treatment

The invention further provides a method for monitoring or evaluating the effect of a treatment to reduce the risk (or likelihood or probability) of pregnancy loss or embryo implantation failure in an individual, wherein the method comprises detecting and/or quantifying the amount of at least one marker gene for decidual cells and at least one marker gene for decidual senescent cells in a biological sample obtained from the individual, and thereby monitoring or evaluating the effect of the treatment, wherein the at least one marker gene for decidual cells comprises PLA2G2A. The relationship between the level of marker genes for decidual cells and decidual senescent cells enables the risk of pregnancy loss or implantation failure to be determined, as described in the preceding section, and thereby allows the determination of whether a treatment is effective in reducing risk. The treatment that may be used to reduce the risk is described further below.

The marker genes and sample that can be used in the method of monitoring or evaluating the effect of treatment may be any as described for use in the preceding sections.

Monitoring or evaluating the effect of treatment to reduce the risk of pregnancy loss or embryo implantation failure includes determining whether the individual is responding or has responded to the treatment, determining the nature of the response, determining the extent of the response, and determining whether or not the individual continues to respond to the treatment in the same way over time. In some cases the individual is determined to be responsive to the treatment or to have had a positive response. Responsiveness or a positive response to treatment means that the individual is expected to derive benefit, or a sufficient extent of benefit, as a result of the treatment. For example, the individual may have or be expected to successfully conceive, or the individual may have an improved prognosis. Non-responsiveness or a negative response to treatment means that the individual is not expected to derive benefit, or a sufficient extent of benefit, from receiving the treatment.

An increased or increasing level of a marker gene for decidual cells e.g. PLA2G2A in a sample, as compared with a reference sample or level, may indicate a positive response to treatment. Alternatively, a decreased or decreasing level of a marker gene for decidual senescent cells e.g. DIO2, as compared with a reference sample or level, may indicate a positive response to treatment. In one instance, an increased or increasing level of a marker gene for decidual cells e.g. PLA2G2A in a sample and a decreased or decreasing level of a marker gene for decidual senescent cells e.g. DIO2, as compared with a reference sample or level, together may indicate a positive response to treatment. The relative abundances of the marker genes for decidual cells and decidual senescent cells may be expressed as a ratio, e.g. a PLA2G2A/DIO2 ratio. The fold-change in this ratio may indicate whether treatment is effective. For instance, an increase in the level of marker genes for decidual cells, and optionally also a decrease in the level of marker genes for decidual senescent cells, would lead to an increased ratio, indicating a positive response to treatment by attenuation of decidual senescence.

A decreased or decreasing or unchanged level of a marker gene for decidual cells e.g. PLA2G2A in a sample, as compared with a reference sample or level, may indicate a negative response to treatment. Alternatively, an increased or increasing or unchanged level of the marker gene for decidual senescent cells e.g. DIO2, as compared with a reference sample or level, may indicate a negative response to treatment. In one instance, decreased or decreasing or unchanged level of the marker gene for decidual cells e.g. PLA2G2A in a sample, and an increased or increasing or unchanged level of the marker gene for decidual senescent cells e.g. DIO2, as compared with a reference sample or level, together may indicate a negative response to treatment. There may be a decrease in the ratio of marker genes for decidual cells to decidual senescent cells caused by a decrease in the level of marker genes for decidual cells, and optionally also an increase in the level of marker genes for decidual senescent cells, indicating a negative response to treatment.

In one instance, the method for monitoring or evaluating the effect of a treatment to reduce the risk of pregnancy loss or embryo implantation failure in an individual further comprises detecting and/or quantifying the level of uNK cells or the level of at least one marker gene for uNK cells in the sample. The detection and/or quantification of the level of uNK cells, for example based on marker genes may be as described in the preceding sections.

An increased or increasing level of the uNK cells or uNK cell gene markers in a sample, as compared with a reference sample, may indicate a positive response to treatment. A decreased or decreasing level of uNK cells or uNK cell gene markers, as compared with a reference sample, may indicate a negative response to treatment.

The above method of treatment of the invention may also further comprise detecting and/or quantifying genes that allow identification of the stage, point or day in the menstrual cycle as described in the preceding sections.

The control sample or reference sample or level may be selected according to any of the criteria described above. In the method of monitoring or evaluating the effect of a treatment to reduce the risk of pregnancy loss or embryo implantation failure in an individual, the level of the marker genes (e.g. PLA2G2A and DIO2) at a first time point before the treatment may be compared with the level of the marker genes (e.g. PLA2G2A and DIO2) at a later time point during or after the treatment. The level of the marker genes (e.g. PLA2G2A and DIO2) during treatment may also be compared with the level of marker genes (e.g. PLA2G2A and DIO2) at a later time point during or after the treatment. In some instances, the level of marker genes may be determined monthly, bi-monthly, every three months, every four months, every five, every six months, every seven months, every eight months, every nine months, every ten months, every eleven months, every twelve months, or at any other suitable time interval as determined by a medical practitioner.

In a related aspect to the above method, the invention also provides a method of preventing or reducing the risk of pregnancy loss or implantation failure in an individual, wherein the at least one marker gene for decidual cells comprises PLA2G2A and wherein the method comprises detecting and/or quantifying the amount of at least one marker gene for decidual cells and at least one marker gene for decidual senescent cells in a biological sample obtained from the individual, and administering an agent or carrying out a treatment regimen effective to prevent or reduce the risk of pregnancy loss or implantation failure in the individual. The agent is administered or the treatment regimen carried out if the marker gene levels are indicative of a risk of pregnancy loss or implantation failure as described above.

### Method of Diagnosing a Reproductive Disorder

The invention additionally provides a method of diagnosing a reproductive disorder in an individual, wherein the method comprises detecting and/or quantifying the amount of at least one marker gene for decidual cells and at least one marker gene for decidual senescent cells in a biological sample obtained from the individual, and thereby diagnosing the disorder, wherein the at least one marker gene for decidual cells comprises PLA2G2A. The marker genes that can be used in the method of diagnosing a reproductive disorder are as described in the preceding sections.

The reproductive disorder may be any reproductive disorder. The reproductive disorder may be any disorder associated with infertility, miscarriage, associated with the risk of obstetric complications or having a negative impact on pregnancy outcome. The reproductive disorder described herein may be any disorder comprising reduced receptivity or failure of the endometrium to be receptive an embryo. Such disorders may include embryo implantation failure, miscarriage, recurrent pregnancy loss or placental disorders. In a preferred embodiment, the reproductive disorder is recurrent pregnancy loss.

Diagnosis includes determining whether or not the individual has a reproductive disorder. Diagnosis may also include determining the particular cause of the reproductive disorder, and determining the different clinical presentations. A positive diagnosis relates to the determination that an individual has the disorder. A negative diagnosis relates to the determination that an individual does not have the disorder.

A decreased or decreasing level of a marker gene for decidual cells e.g. PLA2G2A in a sample, as compared with a reference sample or level, may indicate a positive diagnosis. Alternatively, an increased or increasing level of the marker genes for decidual senescent cells e.g. DIO2, as compared with a reference sample or level, may indicate a positive diagnosis. In one instance, a decreased or decreasing level of the marker gene for decidual cells e.g. PLA2G2A in a sample and an increased or increasing level of the marker gene for decidual senescent cells e.g. DIO2, as compared with a reference sample or level, together may indicate a positive diagnosis.

An unchanged (or similar), increased or increasing level of the marker gene for decidual cells e.g. PLA2G2A in a sample, as compared with a reference sample or level, may indicate a negative diagnosis. Alternatively, an unchanged (or similar), decreased or decreasing level of the marker gene for decidual senescent cells e.g. DIO2, as compared with a reference sample or level, may indicate a negative diagnosis. In one instance, an unchanged (or similar), increased or increasing level of the marker gene for decidual cells e.g. PLA2G2A in a sample, and an unchanged (or similar), decreased or decreasing level of the marker gene for decidual senescent cells e.g. DIO2, as compared with a reference sample or level, together may indicate a negative diagnosis.

In one instance, the method of diagnosing a reproductive disorder in an individual further comprises detecting and/or quantifying the level of uterine natural killer (uNK) cells for example based on the level of at least one marker gene for uNK cells in the sample. The uNK cell marker genes that may be used in the method of diagnosis may be as described in the preceding sections.

A decreased or decreasing level of the uNK cells in a sample, as compared with a reference sample, indicates a positive diagnosis. An unchanged (or similar), increased or increasing level of uNK cells, as compared with a reference sample, indicates a negative diagnosis.

The method of diagnosis of the invention may also further comprise detecting and/or quantifying genes that allow identification of the day in the menstrual cycle as described in the preceding sections.

A control sample or reference sample or level may be provided according to the criteria described above, and may represent a level from an individual or multiple individuals known to have a reproductive disorder, or known to not have any reproductive disorder.

### Methods of Therapy

In accordance with the invention, a method of treating a reproductive disorder in an individual is also provided. The method comprises diagnosing the reproductive disorder according to the method described in the preceding sections and administering an agent or carrying out a treatment regimen effective to treat the reproductive disorder in the individual who is positively diagnosed. Also described is an agent for use in a method of treating a reproductive disorder in an individual, wherein the reproductive disorder is diagnosed according to the methods described in the preceding sections. Also described is the use of an agent for the preparation of a medicament for the treatment of a reproductive disorder. In some embodiments, the individual has an increased level of at least one marker gene for decidual senescent cells e.g. DIO2. In some embodiments, the individual has a decreased level of at least one marker gene for decidual cells, e.g. PLA2G2A. The term “treating” includes a reduction or prevention of the development or progression of the disorder, and the reduction or elimination of an existing disorder or its symptoms. For instance, an individual may be considered treated if the marker levels are altered such that a negative diagnosis may be made. For instance, the relative abundances of the marker genes for decidual cells and decidual senescent cells may be expressed as a ratio, e.g. a PLA2G2A/DIO2 ratio. The fold-change in this ratio may indicate whether treatment is effective. For instance, an increase in the level of marker genes for decidual cells, and optionally also a decrease in the level of marker genes for decidual senescent cells, would lead to an increased ratio, indicating a positive response to treatment by attenuation of decidual senescence. There may also be a decrease in the ratio of marker genes for decidual cells to decidual senescent cells caused by a decrease in the level of marker genes for decidual cells, and optionally also an increase in the level of marker genes for decidual senescent cells, indicating a negative response to treatment.

Agents or treatment regimens that may be administered or carried out may be any agent or treatment regimen known to be effective to treat a reproductive disorder. The agent or treatment regimen may be any able to increase the level of decidual cells and/or uNK cells, and/or decrease the level of decidual senescent cells in the individual. Suitable agents or treatment regimens may include but are not limited to endometrial scratching, dipeptidyl-peptidase IV (DPP4) inhibitors (typically gliptins, e.g. sitagliptin), and senolytic drugs (e.g. dasatinib, quercetin). Examples of DPP4 inhibitors include for instance, vildagliptin, saxagliptin, alogliptin, linagliptin, gemigliptin, evogliptin, omarigliptin, teneligliptin, and are described for example in Deacon CF & Lebovitz HE, Diabetes Obes Metab., 2016; 18(4):333-47. The agent or treatment regimen may target different types of decidual dyshomeostasis, as determined based on the methods of diagnosis previously described. For instance, endometrial scratching may be used to treat decidual failure. Senolytic drugs may be used to treat age-related reproductive disorders. Senolytic drugs or senolytics are drugs that are able to target cellular senescence in order to delay, prevent, alleviate or reverse age-related disorders. The above agents and treatment regimens are also described for use in the methods of reducing the risk of or preventing pregnancy loss or embryo implantation failure described above.

In a preferred aspect, the agent is a DPP4 inhibitor or antagonist. DPP4 is a known marker of glandular differentiation during the midluteal phase of the cycle and is a ubiquitous aminopeptidase expressed both as a cell surface-bound protein and in soluble form (59, 60). DPP4 is also a widely used endometrial receptivity marker gene (61). Stromal cell-derived factor-1α (SDF-1), also known as C-X-C motif chemokine ligand 12 (CXCL12), is a potent chemotactic factor that mediates mobilization of BMDC and homing to the endometrium in response to tissue injury and rising oestradiol levels (62, 63). However, SDF-1 is proteolytically inactivated by DPP4. The inventors have identified that DPP4 inhibitors (gliptins), which are commonly used oral antidiabetic drugs for the treatment of type 2 diabetes (64), may be used to reduce excessive decidual senescence in RPL patients by increasing endometrial stem cells or inhibiting the expression of marker genes for senescent decidual cells e.g. DIO2.

DPP4 inhibitors or antagonists may be any agent that inhibits or antagonises DPP4 expression or activity by any means. The agent may inhibit or antagonise inactivation of SDF-1 by DPP4. Such an agent may be a small molecule, a peptide, a protein, an antibody, a polynucleotide, an oligonucleotide, an antisense RNA, small interfering RNA (siRNA) or small hairpin RNA (shRNA) or any other suitable inhibitor that achieves the function described above. The agent may be a polynucleotide encoding a molecule inhibiting or antagonising DPP4 or may be a polynucleotide, oligonucleotide, antisense RNA, siRNA or shRNA inhibiting expression of DPP4, typically comprising a complementary sequence to DPP4 mRNA and specifically hybridising thereto. An oligonucleotide “specifically hybridises” to a target sequence when it hybridises with preferential or high affinity to the target sequence but does not substantially hybridise, does not hybridise or hybridises with only low affinity to other sequences. More preferably, the oligonucleotide hybridises to the target sequence with a Tₘ that is at least 5° C., at least at least 10° C., at least 20° C., at least 30° C. or at least 40° C., greater than its Tₘ for other nucleic acids. Conditions that permit the hybridisation are well-known in the art (for example, Sambrook et al., 2001, Molecular Cloning: a laboratory manual, 3rd edition, Cold Spring Harbour Laboratory Press; and Current Protocols in Molecular Biology, Chapter 2, Ausubel et al., Eds., Greene Publishing and Wiley-Interscience, New York (1995)). The hybridisation conditions may be stringent conditions as described in the art.

The agent may be an antibody that specifically binds to DPP4 protein or to another protein to inhibit DPP4 function indirectly. An antibody “specifically binds” to a protein when it binds with preferential or high affinity to that protein but does not substantially bind, does not bind or binds with only low affinity to other proteins. For instance, an antibody “specifically binds” a target molecule when it binds with preferential or high affinity to that target but does not substantially bind, does not bind or binds with only low affinity to other human proteins.

An antibody binds with preferential or high affinity if it binds with a Kd of 1×10⁻⁷ M or less, more preferably 5×10⁻⁸ M or less, more preferably 1×10⁻⁸ M or less or more preferably 5×10⁻⁹ M or less. An antibody binds with low affinity if it binds with a Kd of 1×10⁻⁶ M or more, more preferably 1×10⁻⁵ M or more, more preferably 1×10⁻⁴ M or more, more preferably 1×10⁻³ M or more, even more preferably 1×10⁻² M or more.

The antibody may be, for example, a monoclonal antibody, a polyclonal antibody, a single chain antibody, a chimeric antibody, a bispecific antibody, a CDR-grafted antibody or a humanized antibody. The antibody may be an intact immunoglobulin molecule or a fragment thereof such as a Fab, F(ab′)₂ or Fv fragment.

In a preferred embodiment, the agent used in the method of treatment may be a gliptin, for example including sitagliptin, vildagliptin, saxagliptin, linagliptin, gemigliptin, anagliptin, teneligliptin, alogliptin, trelagliptin, omarigliptin, evogliptin, gosogliptin, or dutogliptin. Preferably, the gliptin is sitagliptin.

In some embodiments, the treatment may include a step of detecting an increased level of a marker gene for decidual senescent cells (e.g. DIO2). Where increased DIO2 is detected, the agent used is preferably a DPP4 inhibitor, typically a gliptin and more preferably sitagliptin.

Specific routes, dosages and methods of administration of the therapeutic agents described herein may be routinely determined by the medical practitioner. The agents used in the methods of treatment described herein may be formulated in pharmaceutical compositions. These compositions may comprise, in addition to the therapeutically active ingredient(s), a pharmaceutically acceptable excipient, carrier, diluent, buffer, stabilise or other materials well known to those skilled in the art. Such materials should be non-toxic and should not interfere with the efficacy of the active ingredient. The pharmaceutical carrier or diluent may be, for example, an isotonic solution.

The dose may be determined according to various parameters, especially according to the agent used; the age, weight and condition of the patient to be treated; the route of administration; and the required regimen. Again, a physician will be able to determine the required route of administration and dosage for any particular patient.

The agent can be administered to the patient by any suitable means. The agent can be administered by enteral or parenteral routes such as via oral, buccal, anal, pulmonary, intravenous, intra-arterial, intramuscular, intraosseous, intraperitoneal, intraarticular, topical or other appropriate administration routes. For example, where the agent is a DPP4 inhibitor e.g. sitagliptin, it is preferably administered orally.

A daily dosage for administration of a gliptin such as sitagliptin to a subject such as a human may range from about 50 mg/day to about 2000 mg/day, such as from about 50 mg/day to about 1500 mg/day, from about 50 mg/day to about 100 mg/day, from about 75 mg/day to about 150 mg/day, from about 100 mg/day to about 1500 mg/day, from about 100 mg/day to about 1200 mg/day, from about 100 mg/day to about 175 mg/day, from about 150 mg/day to about 300 mg/day, from about 200 mg/day to about 350 mg/day, from about 250 mg/day to about 400 mg/day, from about 300 mg/day to about 450 mg/day, from about 350 mg/day to about 500 mg/day, from about 400 mg/day to about 550 mg/day, from about 450 mg/day to about 600 mg/day, from about 500 mg/day to about 750 mg/day, from about 600 mg/day to about 800 mg/day, from about 700 mg/day to about 1000mg/day, or from about 800 mg/day to about 1200 mg/day. Preferably, a typical daily dose of sitagliptin is about 100 mg/day or at least about 100 mg/day.

Administration may be in single or multiple doses. Multiple doses may be administered via the same or different routes and to the same or different locations. Alternatively, doses can be via a sustained release formulation, in which case less frequent administration is required. Dosage and frequency may vary depending on the half-life of the agent in the patient and the duration of treatment desired. The dosage as described above may be administered once a day, or may be divided into two doses. The agent may be administered for more than one, for example, at least two or at least three consecutive menstrual cycles. For instance, 100 mg sitagliptin capsules may be taken orally once a day for 2 or 3 consecutive menstrual cycles.

The method of treatment for medical use may comprise administering additional agents known to be effective in treating reproductive disorders to the individual. For instance, progesterone and/or progestogen may be additionally administered.

Also provided herein are methods of preventing pregnancy loss or embryo implantation failure, comprising detecting and/or quantifying the amount of at least one marker gene for decidual cells and at least one marker gene for decidual senescent cells in a biological sample obtained from an individual, to thereby assess the individual as being at risk of pregnancy loss or embryo implantation failure, and administering an agent or carrying out a treatment regimen effective to prevent pregnancy loss or embryo implantation failure, wherein the at least one marker gene for decidual cells comprises PLA2G2A. The agent or treatment regimen may be any agent or treatment regimen described above, preferably administration of a gliptin such as sitagliptin.

Also provided are methods for assessing readiness for conception or successful embryo implantation. Such methods also involve detecting and/or quantifying the amount of at least one marker gene for decidual cells and at least one marker gene for decidual senescent cells in a biological sample obtained from the individual, and thereby assessing the readiness for conception or embryo implantation, wherein the at least one marker gene for decidual cells comprises PLA2G2A. The marker genes and methods of detection that can be used in the above method are as described in the preceding sections.

### Method of Selecting Patients for Treatment

The invention describes a method of selecting patients for treatment to reduce risk (or likelihood or probability) of embryo implantation failure or pregnancy loss in an individual, wherein the method comprises detecting and/or quantifying the amount of at least one marker gene for decidual cells and at least one marker gene for decidual senescent cells in a biological sample obtained from the individual, and thereby selecting the patients for treatment to reduce the risk of pregnancy loss or embryo implantation failure based on the level of the marker genes, wherein the at least one marker gene for decidual cells comprises PLA2G2A. The method may include detecting and/or quantifying the level of uNK cells in the sample or uNK cell gene markers in a sample. The marker genes that can be used in the method of selecting patients are as those described in the preceding sections. The treatment may be with any treatment regimen or agent as described above.

An individual in which an increased level of at least one marker gene for decidual senescent cells e.g. DIO2 is detected may be selected as a patient for treatment. An individual in which a decreased level of at least one marker gene for decidual cells e.g. PLA2G2A may also be selected as a patient for treatment. The selected patient is preferably treated with a DPP4 inhibitor. Preferably, the marker gene for decidual senescent cells is DIO2 and the selected patient is treated with sitagliptin.

The method of the invention may also further comprise detecting and/or quantifying genes that allow identification of the day in the menstrual cycle as described in the preceding sections.

The relationship between the level of marker genes for decidual cells, decidual senescent cells and uNK cells may enable a particular defect in the decidual pathway to be determined. It can then be determined whether a patient would likely benefit from selection of a particular type of treatment to reduce risk of embryo implantation failure or miscarriage.

### Method of Stratifying Patients

The invention further provides a method of stratifying patients into different groups, for instance for clinical studies. The method comprises detecting and/or quantifying the amount of at least one marker gene for decidual cells and at least one marker gene for decidual senescent cells in a biological sample obtained from an individual, wherein the at least one marker gene for decidual cells comprises PLA2G2A. The method may include detecting and/or quantifying the level of uNK cells in the sample for example based on the level of uNK cell gene markers in a sample. The marker genes that may be used in the method of stratifying patients may be any as described in the preceding sections.

The method of the invention may also further comprise detecting and/or quantifying genes that allow identification of the day in the menstrual cycle as described in the preceding sections.

The relationship between the level of marker genes for decidual cells, decidual senescent cells and uNK cells may enable the particular defect in the decidual pathway to be determined. Patients having different patterns or levels of these markers can then be grouped accordingly for clinical studies.

### Kits

The invention further provides a kit, which may be suitable for use in any method of the invention. The kit may include means (e.g. reagents) for detecting and/or quantifying at least one marker gene for decidual cells and at least one marker gene for decidual senescent cells at a nucleic acid or protein level in a biological sample from an individual, wherein the at least one marker gene for decidual cells comprises PLA2G2A. The kit thus includes reagents for detecting and/or quantitating PLA2G2A. The at least one marker gene for decidual senescent cells may comprise DIO2. The at least one marker gene for decidual senescent cells may be selected from DIO2, CLU and IGFBP1. The kit may also comprise means for detecting and/or quantifying the level of UNK cells or uNK cell markers. The marker genes that may be detected using the kit may be any of those described in the preceding sections. The kit may also further comprise means for detecting and/or quantifying genes that allow identification of the day in the menstrual cycle as described in the preceding sections. Preferably, the genes that allow identification of the timing of the day in the menstrual cycle comprise, consist or consist essentially of GPX3 and SLC15A2.

The only reagents for detecting and/or quantifying marker genes comprised in the kit may be reagents for detection of the marker genes specified above.

The kit may additionally include instructions for use of the kit in accordance with methods of the invention. The kit may also comprise details regarding which individuals the method may be carried out upon. The kit may also be provided with means for obtaining an endometrial biopsy sample. The kit may also comprise a test requisition form with details to be sent to the analysers. The kit may additionally comprise means for the measurement of other laboratory or clinical parameters, and/or a container for holding a biological sample isolated from a subject.

The kit may additionally comprise one or more other reagents or instruments which enable the method to be carried out. Such reagents or instruments may include one or more of the following: suitable buffer(s) (aqueous solutions), calibration curve standards, developing reagents, enzymes, labels, reacting surfaces, means for detection, control samples, standards, instructions, interpretive information, means to isolate a relevant biomarker from a sample, means to obtain a sample from the individual (such as a vessel or an instrument comprising a needle) or a support comprising wells on which quantitative reactions can be done.

In one instance, the kit may comprise a cryotube and RNA stabilizing solution.

In one instance, use of the above test kit for assessing pregnancy loss or embryo implantation failure, or for diagnosing a reproductive disorder is described. The use may comprise steps as described above in relation to the methods of the invention detecting marker genes. In a preferred embodiment, the kit is used to assessing the risk of, or diagnosing, recurrent pregnancy loss.

### EXAMPLES

### Materials and Methods

### Ethical Approval and Sample Collection

The study was approved by the NHS National Research Ethics-Hammersmith and Queen Charlotte's & Chelsea Research Ethics Committee (1997/5065). All samples were obtained with written informed consent and in accordance with The Declaration of Helsinki (2000) guidelines. Human endometrial biopsies were obtained from women attending the Implantation Clinic, a dedicated research clinic at University Hospitals Coventry and Warwickshire (UHCW) National Health Service Trust. Surplus tissue from endometrial biopsies obtained for diagnostic purposes at the Implantation Research Clinic was used for this study. Samples were obtained during the luteal phase of ovulatory, non-hormonally stimulated menstrual cycles, timed relative to the pre-ovulatory LH surge, using a Wallach Endocell™ endometrial sampler. Overt uterine pathology was excluded by transvaginal ultrasound scan prior to the biopsy.

### Drop-Seq Analysis of Timed Endometrial Biopsies

Six LH-timed endometrial biopsies were processed as described in detail elsewhere (Lucas et al., 2020). After tissue digestion, red blood cells were removed from the flow-through by Ficoll density gradient centrifugation. Single-cell fractions were then subjected to Drop-Seq analysis.

### Reverse Transcription Quantitative PCR (RT-qPCR)

RNA was extracted from endometrial biopsies which had been placed in RNA Later in the clinic (<1 min after collection), using RNeasy plus Universal mini kit (QIAGEN) according to the manufacturer's instructions. Reverse transcription was performed from 1 μg RNA using the Quantitect Reverse Transcription Kit (QIAGEN) and cDNA was diluted to 10 ng/μl equivalent before use in qPCR. Amplification was performed on a QuantStudio 5 (ThermoFisher) in 10 μl reactions using 2×Quantifast SYBR Green RT-PCR Kit (QIAGEN), with 300 nM each of forward and reverse primers. Primer sequences were as follows: DIO2 forward: 5′-ACT CGG TCA TTC TGC TCA A-3′, DIO2 reverse: 5′-TTC CAG ACG CAG CGC AGT-3′, PLA2G2A forward 5′ AAA GGA AGC CGC ACT CAG TT-3′, PLA2G2A reverse: 5′-TTT CCA GGG AAG AGG GGA C-3′. Centile calculations were performed on dCt values using R v3.5 software.

### Multiplexed Single Molecule in Situ Hybridization (RNAScope)

Formalin-fixed paraffin-embedded (FFPE) samples were cut to 5 μm sections. RNA in situ hybridization was carried with RNAscope® 2.5 HD Duplex Reagent Kit (ACD, California, USA) with probes for PLA2G2A (581101-C2) and DIO2 (562211) according to manufacturer's guidelines. Following hybridization and amplification, slides were counterstained with 50% haematoxylin. Images were obtained using Mirax Midi slide scanner using a 20× objective lens and opened in Panoramic Viewer v1.15.4 (3DHISTECH Ltd) for analysis.

### Spatial Transcriptomics

Endometrial biopsies were fixed overnight in 10% neutral buffered formalin at 4° C. and wax embedded in Surgipath Formula ‘R’ paraffin using the Shandon Excelsior ES Tissue processor (ThermoFisher). 4 endometrial biopsies were selected on basis of morphology, PLA2G2A/DIO2 expression and RNA integrity (DV.200 >50). 5 um sections were prepared, deparaffinized and stained with haematoxylin and eosin according to the protocol (10× Genomics). Spatial gene expression slides and reagents kits were used according to manufacturer instructions. Each capture area (6.5×6.5 mm2) contains 5,000 barcoded spots that are 55 μm in diameter (100 μm centre to centre between spots) providing an average resolution of 1 to 10 cells). Eluted libraries were analysed using Agilent Bioanalyzer High Sensitivity DNA chip to assess quality and determine library size. Library dilution and denaturation was performed as per standard Illumina protocols and sequenced using NextSeq 500/550 High Output kit v2.5 (150 cycles). Sequencing was performed with the recommended 10× protocol (read 1:28 cycles; i7 index read: 10 cycles; i5 index read: 10 cycles; and read 2: 91 cycles), yielding between 21 million and 50 million sequenced reads. Reads were processed using Spaceranger software v1.3.0 with refdata-gex-GRCh38-2020-A as reference genomic data and analysed in R v4.1.3 using Seurat v4.0.4.

### Bulk RNA-Sequencing

Total RNA was extracted from endometrial biopsies using RNeasy plus Universal mini kit (QIAGEN) according to the manufacturer's instructions. Library dilution and denaturation was performed as per standard Illumina protocols and sequenced using NextSeq 500/550 High Output kit v2.5 (75 cycles). Reads were mapped to the GRCh38 human genome assembly and gencode v38 annotation using STAR v2.7.9. Reads overlapping gene regions were counted using HTSeq v0.6.1 with “intersection-nonempty” option. Differential gene expression analysis was performed with DESeq2 v1.34.0 in R.

### SIMPLANT Clinical Trial Data

Details regarding methods and governance of the SIMPLANT clinical trial can be found in Tewary et al., 2020. Briefly, a double-blind, randomised, placebo-controlled feasibility trial on women aged 18 to 42 years with a history of 3 or more miscarriages was trialled. Thirty-eight subjects were randomised to either the DPP4 inhibitor sitagliptin (100 mg daily) for 3 consecutive cycles or identical placebo capsules. Tissue samples were subjected to explorative investigations.

### RESULTS

### 1. Discovery of a New Biomarker of Anti-Inflammatory Decidual Cells

Using high-throughput single-cell droplet barcoding of the transcriptomic changes along the decidual pathway in vitro, we defined the characteristics of the diverging decidual subpopulations. Single-cell RNA sequencing analysis luteal phase endometrial biopsies initially identified two putative biomarkers, SCARA5 and DIO2, as selective marker genes for decidual cells and stressed/senescent cells, respectively (Lucas et al., 2020; WO 2021/032973).

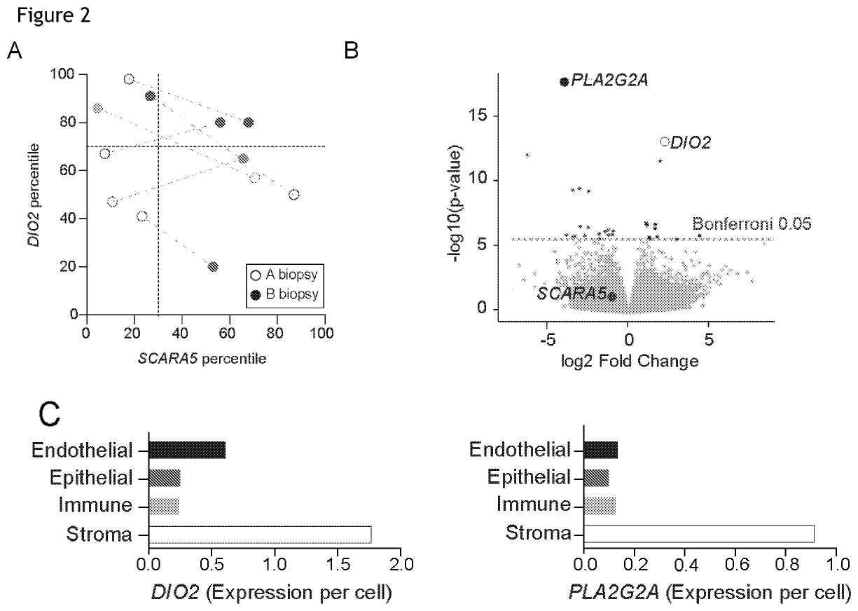

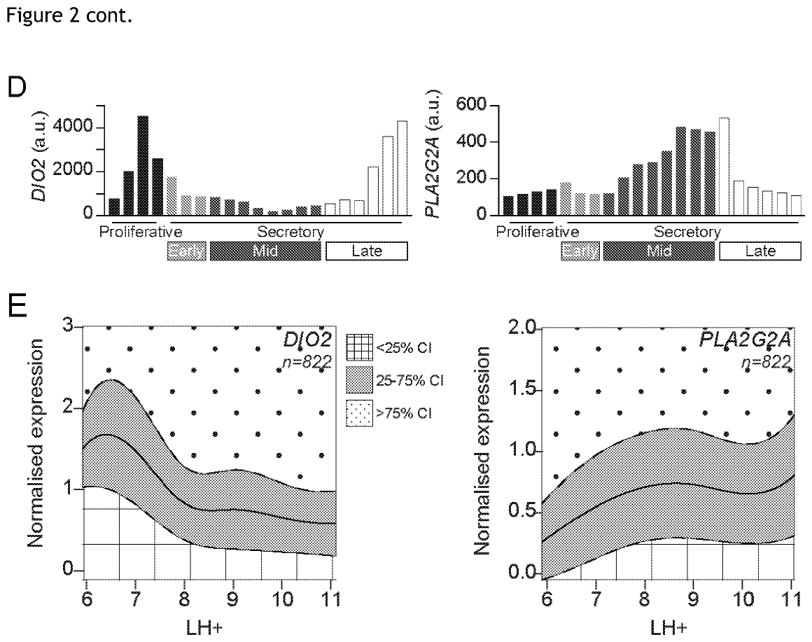

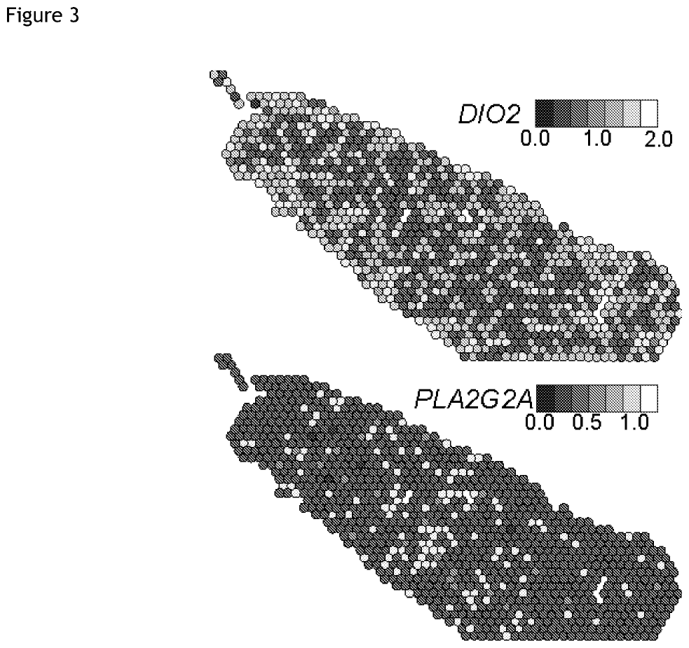

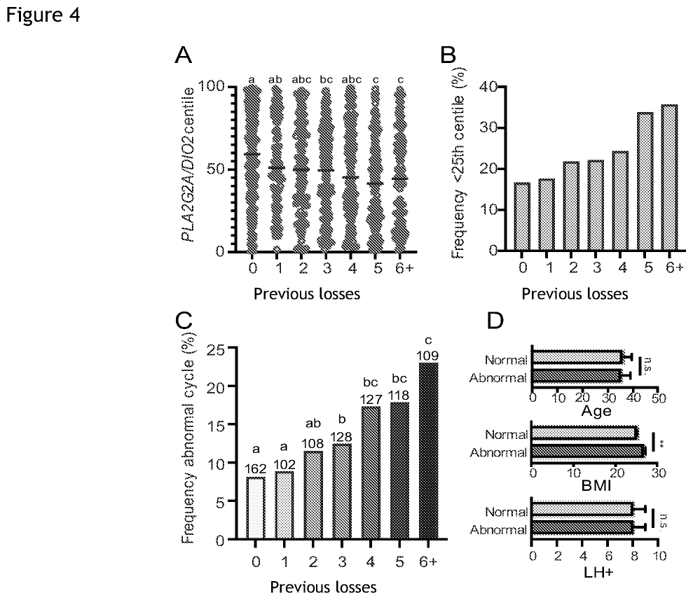

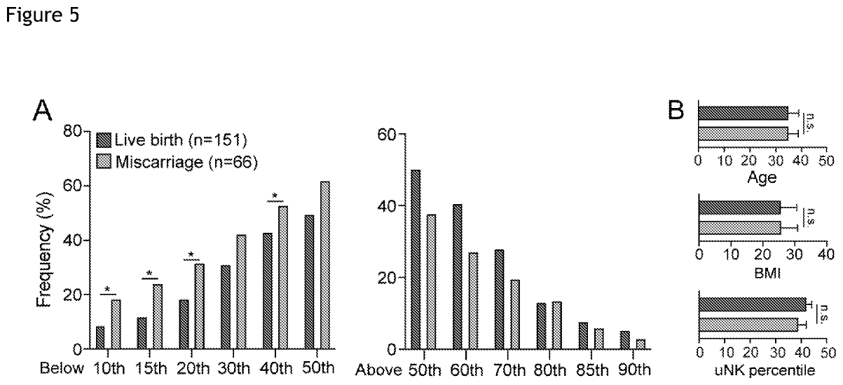

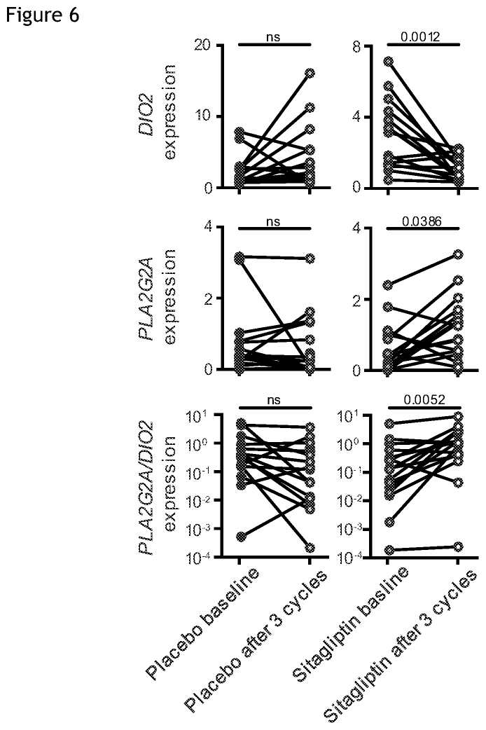

Further bulk RNA sequencing (RNA-seq) of paired biopsies, that is, obtained in the same patient but different cycles, led to the discovery of the biomarker gene PLA2G2A, encoding Phospholipase A2 Group IIA (FIGS. 2A and 2B). Like SCARA5, PLA2G2A is a stromal cell-specific biomarker gene of progesterone-dependent decidual cells but with a much greater dynamic range in expression levels, rendering it a significantly more sensitive biomarker. Both DIO2 and PLA2G2A are highly enriched within the endometrial stroma (FIG. 2C), although they display contrasting temporal regulation across the menstrual cycle (FIG. 2D). We created a reference range of expression of both genes in peri-implantation endometrium by RTQ-PCR analysis of 822 biopsy samples obtained 6 to 11 days after the preovulatory luteinising hormone (LH+6 to LH+11) surge as determined by over-the-counter home ovulation test kits (FIG. 2E). Percentiles are used to compare the relative expression of biomarkers in endometrial samples obtained on different days in the menstrual cycle.

We elucidated the spatial organization of PLA2G2A- and DIO2-expressing stromal subpopulations by multiplexed single-molecule in situ hybridization (RNAScope®) and Visium spatial transcriptomics (10× Genomics). Spatial analyses confirmed that DIO2 and PLA2G2A marked distinct stromal subpopulations in peri-implantation endometrium (FIG. 3). Further, while DIO2-positive cells were enriched in proximity of the luminal epithelium (which lines the uterine cavity), PLA2G2A-positive cells resided deeper into the tissue. Thus, quantification of DIO2 and PLA2G2A transcript levels in endometrial samples provides information on the spatial organization of the tissue during the peri-implantation window.

### 2. Diagnostic and Prognostic Potential of Normalised PLA2G2A/DIO2 Expression Ratios in Luteal-Phase Endometrial Biopsies in Recurrent Pregnancy Loss

We analysed the PLA2G2A DIO2 ratio (expressed as percentiles of ratio) in 854 LH-timed endometrial biopsies of women with a history of 0 to 18 prior miscarriages. The lower the ratio of these marker genes, the higher the relative excess of stressed/senescent cells over anti-inflammatory decidual cells in a sample, and vice versa. As shown in FIG. 4A, the median PLA2G2A/DIO2 ratio decreases stepwise in function of the number of previous miscarriages in this sample set. The frequency of samples with a ratio below the 25^{th} percentile (lower quartile) decreased, whereas the frequency of samples with a ratio above the 75^{th} percentile (upper quartile) increased stepwise with each additional loss (FIG. 4B).

A cut-off PLA2G2A/DIO2 ratio <15^{th} percentile maximised the separation of the samples from women without a history of prior pregnancy loss and women with 6 or more previous miscarriages (FIG. 4C). Importantly, neither maternal age or the timing of the biopsy relative to the preovulatory LH surge differed significantly between samples below or above the 15^{th} percentile cut-off ratio (FIG. 4D). However, patients below the 5^{th} percentile cut-off ratio had a significantly higher body mass index (BMI) (P<0.001), which is a well-documented risk factor of recurrent miscarriage/recurrent pregnancy loss (Quenby et al., 2021).

We also analysed 217 endometrial biopsies obtained prior to a subsequent pregnancy. No restrictions were placed on the time interval between the day of biopsy and the start of the pregnancy. As shown in FIG. 5A, recurrent miscarriage patients with low PLA2G2A/DIO2 ratios were significantly more likely to miscarry again (<20 percentile). Conversely, the patients with high PLA2G2A/DIO2 ratios (>60 percentile) were less likely to miscarry again. Importantly, maternal age, body mass index (BMI), and uNK cell levels did not differ significantly (P>0.05) between patients who following the endometrial biopsy had a live birth or pregnancy loss (FIG. 5B).

### 3. Therapeutic Intervention: Sitagliptin Treatment Improves the PLA2G2A/DIO2 Expression Ratios in Recurrent Pregnancy Loss

We previously reported that sitagliptin, a dipeptidyl-peptidase IV (DPP4) inhibitor used in the management of type 2 diabetes, increases the recruitment of bone marrow-derived MSCs when given over 3 menstrual cycles (Tewary et al., 2020). We used the endometrial samples obtained during this double-blinded placebo-controlled randomised pilot trial to examine the impact of sitagliptin versus placebo on endometrial PLA2G2A/DIO2 ratios before and after the trial. As shown in FIG. 6, patients who received sitagliptin significantly improved their endometrial PLA2G2A/DIO2 ratios (P=0.0052). By contrast, the endometrial PLA2G2A/DIO2 ratios were not significantly different before and after the trial (P>0.05) in the placebo group.

### REFERENCES

1 Rai, R. & Regan, L. Recurrent miscarriage. Lancet 368, 601-611, doi: 10.1016/S0140-6736 (06) 69204-0 (2006).
2 Hardy, K., Hardy, P. J., Jacobs, P. A., Lewallen, K. & Hassold, T. J. Temporal changes in chromosome abnormalities in human spontaneous abortions: Results of 40 years of analysis. Am J Med Genet A 170, 2671-2680, doi: 10.1002/ajmg.a.37795 (2016).
3 Hassold, T. et al. A cytogenetic study of 1000 spontaneous abortions. Ann Hum Genet 44, 151-178 (1980).
4 Ogasawara, M., Aoki, K., Okada, S. & Suzumori, K. Embryonic karyotype of abortuses in relation to the number of previous miscarriages. Fertility and sterility 73, 300-304 (2000).
5 Stephenson, M. D., Awartani, K. A. & Robinson, W. P. Cytogenetic analysis of miscarriages from couples with recurrent miscarriage: a case-control study. Hum Reprod 17, 446-451 (2002).
6 Sullivan, A. E., Silver, R. M., LaCoursiere, D. Y., Porter, T. F. & Branch, D. W. Recurrent fetal aneuploidy and recurrent miscarriage. Obstet Gynecol 104, 784-788, doi: 10.1097/01.AOG.0000137832.86727.e2 (2004).
7 Carp, H. et al. Karyotype of the abortus in recurrent miscarriage. Fertil Steril 75, 678-682 (2001).
8 Robberecht, C. et al. Cytogenetic and morphological analysis of early products of conception following hystero-embryoscopy from couples with recurrent pregnancy loss. Prenat Diagn 32, 933-942, doi: 10.1002/pd.3936 (2012).
9 ESHRE. Recurrent Pregnancy Loss: A Guideline of the European Society of Human Reproduction and Embryology. (2017).
10 Practice Committee of the American Society for Reproductive, M. Evaluation and treatment of recurrent pregnancy loss. Fertility and Sterility 5, 1103-1111 (2012).
11 Cha, J., Sun, X. & Dey, S. K. Mechanisms of implantation: strategies for successful pregnancy. Nature medicine 18, 1754-1767, doi: 10.1038/nm.3012 (2012).
12 Moraes, J. G. N. et al. Uterine influences on conceptus development in fertility-classified animals. Proc Natl Acad Sci USA 115, E1749-E1758, doi: 10.1073/pnas. 1721191115 (2018).
13 Salker, M. S. et al. Disordered IL-33/ST2 activation in decidualizing stromal cells prolongs uterine receptivity in women with recurrent pregnancy loss. PLoS One 7, e52252, doi: 10.1371/journal.pone.0052252 (2012).
14 Gellersen, B. & Brosens, J. J. Cyclic decidualization of the human endometrium in reproductive health and failure. Endocrine reviews 35, 851-905, doi: 10.1210/er.2014-1045 (2014).
15 Brighton, P. J. et al. Clearance of senescent decidual cells by uterine natural killer cells in cycling human endometrium. eLife 6, doi: 10.7554/eLife.31274 (2017).
16 Nancy, P. et al. Chemokine gene silencing in decidual stromal cells limits T cell access to the maternal-fetal interface. Science 336, 1317-1321, doi: 10.1126/science. 1220030 (2012).
17 Macosko, E. Z. et al. Highly Parallel Genome-wide Expression Profiling of Individual Cells Using Nanoliter Droplets. Cell 161, 1202-1214, doi: 10.1016/j.cell.2015.05.002 (2015).
18 Lucas, E. S. et al. Loss of Endometrial Plasticity in Recurrent Pregnancy Loss. Stem Cells 34, 346-356, doi: 10.1002/stem.2222 (2016).
19 Lucas, E. S., Vrljicak, P., Muter, J., Diniz-Da-Costa, M. M., Brighton, P. J., Kong, C. S., Lipecki, J., Fishwick, K. J., Odendaal, J., Ewington, L. J., Quenby, S., Ott, S. & Brosens, J. J. Recurrent pregnancy loss is associated with a pro-senescent decidual response during the peri-implantation window. Commun Biol, 3, 37. (2020)
20 Tewary, S., Lucas, E. S., Fujihara, R., Kimani, P. K., Polanco, A., Brighton, P. J., Muter, J., Fishwick, K. J., Da Costa, M., Ewington, L. J., Lacey, L., Takeda, S., Brosens, J. J. & Quenby, S. Impact of sitagliptin on endometrial mesenchymal stem-like progenitor cells: A randomised, double-blind placebo-controlled feasibility trial. EBioMedicine, 51, 102597. (2020)
21 Magnus, M. C., Wilcox, A. J., Morken, N.-H., Weinberg, C. R. & Håberg, S. E. Role of maternal age and pregnancy history in risk of miscarriage: Prospective register based study. bmj, 364. (2019)
22 Brosens, J. J., Salker, M. S., Teklenburg, G., Nautiyal, J., Salter, S., Lucas, E. S., Steel, J. H., Christian, M., Chan, Y. W., Boomsma, C. M., Moore, J. D., Hartshorne, G. M., Sucurovic, S., Mulac-Jericevic, B., Heijnen, C. J., Quenby, S., Koerkamp, M. J., Holstege, F. C., Shmygol, A. & Macklon, N. S. Uterine selection of human embryos at implantation. Sci Rep, 4, 3894. (2014).
23 Ewington, L. J., Tewary, S. & Brosens, J. J. New insights into the mechanisms underlying recurrent pregnancy loss. J Obstet Gynaecol Res 45, 258-265, doi: 10.1111/jog. 13837 (2019).
24 Brosens, J. J., Bennett, P. R., Abrahams, V. M., Ramhorst, R., Coomarasamy, A., Quenby, S., Lucas, E. S. & Mccoy, R. C. Maternal selection of human embryos in early gestation: Insights from recurrent miscarriage. Semin Cell Dev Biol. (2022)
25 Al-Sabbagh, M. et al. NADPH oxidase-derived reactive oxygen species mediate decidualization of human endometrial stromal cells in response to cyclic AMP signaling. Endocrinology 152, 730-740, doi: 10.1210/en.2010-0899 (2011).
26 Zhong J, Rajagopalan S. Dipeptidyl Peptidase-4 Regulation of SDF-1/CXCR4 Axis: Implications for Cardiovascular Disease. Front Immunol. 2015; 6:477.
27 Imai K, Maeda M, Fujiwara H, Kariya M, Takakura K, Kanzaki H, et al. Dipeptidyl peptidase IV as a differentiation marker of the human endometrial glandular cells. Hum Reprod. 1992; 7 (9):1189-94.
28 Suhorutshenko M, Kukushkina V, Velthut-Meikas A, Altmae S, Peters M, Magi R, et al. Endometrial receptivity revisited: endometrial transcriptome adjusted for tissue cellular heterogeneity. Hum Reprod. 2018; 33(11):2074-86.
29 Wang X, Mamillapalli R, Mutlu L, Du H, Taylor HS. Chemoattraction of bone marrow-derived stem cells towards human endometrial stromal cells is mediated by estradiol regulated CXCL12 and CXCR4 expression. Stem Cell Res. 2015; 15(1):14-22.
30 Yi KW, Mamillapalli R, Sahin C, Song J, Tal R, Taylor HS. Bone marrow-derived cells or C-X-C motif chemokine 12 (CXCL12) treatment improve thin endometrium in a mouse model. Biol Reprod. 2018.
31 Deacon CF. A review of dipeptidyl peptidase-4 inhibitors. Hot topics from randomized controlled trials. Diabetes Obes Metab. 2018; 20 Suppl 1:34-46.
32 Quenby, S., Gallos, I. D., Dhillon-Smith, R. K., Podesek, M., Stephenson, M. D., Fisher, J., Brosens, J. J., Brewin, J., Ramhorst, R., Lucas, E. S., Mccoy, R. C., Anderson, R., Daher, S., Regan, L., Al-Memar, M., Bourne, T., Macintyre, D. A., Rai, R., Christiansen, O. B., Sugiura-Ogasawara, M., Odendaal, J., Devall, A. J., Bennett, P. R., Petrou, S. & Coomarasamy, A. 2021. Miscarriage matters: the epidemiological, physical, psychological, and economic costs of early pregnancy loss. Lancet, 397, 1658-1667.
33 Erkenbrack, E. M. et al. The mammalian decidual cell evolved from a cellular stress response. PLoS Biol 16, e2005594, doi: 10.1371/journal.pbio.2005594 (2018).
34 Kuroda, K. et al. Elevated periimplantation uterine natural killer cell density in human endometrium is associated with impaired corticosteroid signaling in decidualizing stromal cells. The Journal of clinical endocrinology and metabolism 98, 4429-4437, doi: 10.1210/jc.2013-1977 (2013).
35 Song, J. J. et al. Role of glutaredoxin in metabolic oxidative stress. Glutaredoxin as a sensor of oxidative stress mediated by H2O2. J Biol Chem 277, 46566-46575, doi: 10.1074/jbc.M206826200 (2002)
36 Zuo, R. J. et al. Crystallin alphaB acts as a molecular guard in mouse decidualization: regulation and function during early pregnancy. FEBS Lett 588, 2944-2951, doi: 10.1016/j.febslet.2014.05.045 (2014).
37 Latini, F. R. et al. ABI3 ectopic expression reduces in vitro and in vivo cell growth properties while inducing senescence. BMC Cancer 11, 11, doi: 10.1186/1471-2407 Nov. 11 (2011).
38 Michishita, E., Garces, G., Barrett, J. C. & Horikawa, I. Upregulation of the KIAA1199 gene is associated with cellular mortality. Cancer Lett 239, 71-77, doi: 10.1016/j.canlet.2005.07.028 (2006).
39 Petropoulou, C., Trougakos, I. P., Kolettas, E., Toussaint, O. & Gonos, E. S. Clusterin/apolipoprotein J is a novel biomarker of cellular senescence that does not affect the proliferative capacity of human diploid fibroblasts. FEBS Lett 509, 287-297 (2001).
40 Trougakos, I. P. The molecular chaperone apolipoprotein J/clusterin as a sensor of oxidative stress: implications in therapeutic approaches—a mini-review. Gerontology 59, 514-523, doi: 10.1159/000351207 (2013).
41 Bianco, A. C. & Kim, B. W. Deiodinases: implications of the local control of thyroid hormone action. J Clin Invest 116, 2571-2579, doi: 10.1172/JCI29812 (2006).
42 Altmae, S. et al. Meta-signature of human endometrial receptivity: a meta-analysis and validation study of transcriptomic biomarkers. Sci Rep 7, 10077, doi: 10.1038/s41598-017-10098-3 (2017).
43 Vento-Tormo, R. et al. Single-cell reconstruction of the early maternal-fetal interface in humans. Nature 563, 347-353, doi: 10.1038/s41586-018-0698-6 (2018).
44 Drury, J. A., Tang, A. W., Turner, M. A. & Quenby, S. A rapid, reliable method for uNK cell density estimation. J Reprod Immunol 97, 183-185, doi: 10.1016/j.jri.2012.12.002 (2013).
45 Ramsey, E. M., Houston, M. L. & Harris, J. W. Interactions of the trophoblast and maternal tissues in three closely related primate species. American journal of obstetrics and gynecology 124, 647-652 (1976).
46 Emera, D., Romero, R. & Wagner, G. The evolution of menstruation: a new model for genetic assimilation: explaining molecular origins of maternal responses to fetal invasiveness. Bioessays 34, 26-35, doi: 10.1002/bies.201100099 (2012).
47 Evans, J. & Salamonsen, L. A. Inflammation, leukocytes and menstruation. Rev Endocr Metab Disord 13, 277-288, doi: 10.1007/s11154-012-9223-7 (2012).
48 Evans, J. & Salamonsen, L. A. Decidualized human endometrial stromal cells are sensors of hormone withdrawal in the menstrual inflammatory cascade. Biol Reprod 90, 14, doi: 10.1095/biolreprod. 113.108175 (2014).
50 O′Leary, M. A. et al. The placental mammal ancestor and the post-K-Pg radiation of placentals. Science 339, 662-667, doi: 10.1126/science. 1229237 (2013).
51 Marcais, A. et al. The metabolic checkpoint kinase mTOR is essential for IL-15signaling during the development and activation of NK cells. Nat Immunol 15, 749-757, doi: 10.1038/ni.2936 (2014).
52 Mokhtar, N. M. et al. Progestin regulates chemokine (C-X-C motif) ligand 14 transcript level in human endometrium. Molecular human reproduction 16, 170-177, doi: 10.1093/molehr/gap100 (2010).
53 Kane, N., Kelly, R., Saunders, P. T. & Critchley, H. O. Proliferation of uterine natural killer cells is induced by human chorionic gonadotropin and mediated via the mannose receptor. Endocrinology 150, 2882-2888, doi: 10.1210/en.2008-1309 (2009).
54 Kao, L. C. et al. Global gene profiling in human endometrium during the window of implantation. Endocrinology 143, 2119-2138, doi: 10.1210/endo. 143.6.8885 (2002).
55 Gibson, D. A., Greaves, E., Critchley, H. O. & Saunders, P. T. Estrogen-dependent regulation of human uterine natural killer cells promotes vascular remodelling via secretion of CCL2. Hum Reprod 30, 1290-1301, doi: 10.1093/humrep/dev067 (2015).
56 Santamaria, X., Mas, A., Cervello, I., Taylor, H. & Simon, C. Uterine stem cells: from basic research to advanced cell therapies. Hum Reprod Update 24, 673-693, doi: 10.1093/humupd/dmy028 (2018).
57 Murakami, K. et al. Deficiency in clonogenic endometrial mesenchymal stem cells in obese women with reproductive failure—a pilot study. PloS one 8, e82582, doi: 10.1371/journal.pone.0082582 (2013).
58 Castellana, B. et al. Maternal obesity alters uterine NK activity through a functional KIR2DL1/S1 imbalance. Immunol Cell Biol 96, 805-819, doi: 10.1111/imcb.12041 (2018).
59 Perdu, S. et al. Maternal obesity drives functional alterations in uterine NK cells. JCI Insight 1, e85560, doi: 10.1172/jci.insight.85560 (2016).
60 Boots, C. E., Bernardi, L. A. & Stephenson, M. D. Frequency of euploid pregnancy lossis increased in obese women with recurrent early pregnancy loss. Fertil Steril 102, 455-459, doi: 10.1016/j.fertnstert.2014.05.005 (2014).
61 Leitao, B. et al. Silencing of the JNK pathway maintains progesterone receptor activity in decidualizing human endometrial stromal cells exposed to oxidative stress signals. FASEB journal: official publication of the Federation of American Societies for Experimental Biology 24, 1541-1551 (2010).
62 Leitao, B. B., Jones, M. C. & Brosens, J. J. The SUMO E3-ligase PIAS1 couples reactive oxygen species-dependent JNK activation to oxidative cell death. FASEB J 25, 3416-3425, doi: 10.1096/fj.11-186346 (2011).
63 Salker, M. S. et al. Deregulation of the serum-and glucocorticoid-inducible kinase SGK1 in the endometrium causes reproductive failure. Nature medicine 17, 1509-1513, doi: 10.1038/nm.2498 (2011).
64 Kajihara, T. et al. Differential expression of FOXO1 and FOXO3a confers resistance to oxidative cell death upon endometrial decidualization. Mol Endocrinol 20, 2444-2455, doi: 10.1210/me.2006-0118 (2006).
65 Muter, J. et al. Progesterone-Dependent Induction of Phospholipase C-Related Catalytically Inactive Protein 1 (PRIP-1) in Decidualizing Human Endometrial Stromal Cells. Endocrinology 157, 2883-2893, doi: 10.1210/en.2015-1914 (2016).
66 Kuroda, K. et al. Elevated periimplantation uterine natural killer cell density in human endometrium is associated with impaired corticosteroid signaling in decidualizing stromal cells. The Journal of clinical endocrinology and metabolism 98, 4429-4437, doi: 10.1210/jc.2013-1977 (2013).
67 Weyemi, U. et al. ROS-generating NADPH oxidase NOX4 is a critical mediator in oncogenic H-Ras-induced DNA damage and subsequent senescence. Oncogene 31, 1117-1129, 10 doi: 10.1038/onc.2011.327 (2012).
68 Colombo, A. R., Elias, H. K. & Ramsingh, G. Senescence induction universally activates transposable element expression. Cell Cycle, 1-12, doi: 10.1080/15384101.2018.1502576 (2018).
69 van Deursen, J. M. The role of senescent cells in ageing. Nature 509, 439-446, doi: 10.1038/nature13193 (2014).
70 Uhlen, M. et al. Proteomics. Tissue-based map of the human proteome. Science 347, 1260419, doi: 10.1126/science. 1260419 (2015).
71 van Deursen JM. Senolytic therapies for healthy longevity. Science, 364, 6441:636-7 (2019).
72 Barros FS, Brosens, J. J., Brighton, P. J. Isolation and Primary Culture of Various Cell Types from Whole Human Endometrial Biopsies. Bio-protocol. 2016; 6:e2028.
73 Masuda H, Anwar SS, Buhring HJ, Rao JR, Gargett CE. A novel marker of human endometrial mesenchymal stem-like cells. Cell Transplant. 2012; 21(10):2201-14.
74 Lucas ES, Dyer NP, Murakami K, Lee YH, Chan YW, Grimaldi G, et al. Loss of Endometrial Plasticity in Recurrent Pregnancy Loss. Stem Cells. 2016; 34(2):346-56.
75 Pfaffl MW. A new mathematical model for relative quantification in real-time RT-PCR. Nucleic Acids Res. 2001; 29(9):e45.
76 Turco MY, Gardner L, Hughes J, Cindrova-Davies T, Gomez MJ, Farrell L, et al. Long-term, hormone-responsive organoid cultures of human endometrium in a chemically defined medium. Nat Cell Biol. 2017.
77 Lash GE, Bulmer JN, Li TC, Innes BA, Mariee N, Patel G, et al. Standardisation of uterine natural killer (uNK) cell measurements in the endometrium of women with recurrent reproductive failure. J Reprod Immunol. 2016; 116:50-9.
78 Murakami K, Bhandari H, Lucas ES, Takeda S, Gargett CE, Quenby S, et al. Deficiency in clonogenic endometrial mesenchymal stem cells in obese women with reproductive failure--a pilot study. PLoS One. 2013; 8(12):e82582.
79 Wang W, Choi BK, Li W, Lao Z, Lee AY, Souza SC, et al. Quantification of intact and truncated stromal cell-derived factor-1 alpha in circulation by immunoaffinity enrichment and tandem mass spectrometry. J Am Soc Mass Spectrom. 2014; 25(4):614-25.
80 Du H, Taylor HS. Contribution of bone marrow-derived stem cells to endometrium and endometriosis. Stem Cells. 2007; 25(8):2082-6.
81 Morelli SS, Rameshwar P, Goldsmith LT. Experimental evidence for bone marrow as a source of nonhematopoietic endometrial stromal and epithelial compartment cells in a murine model. Biol Reprod. 2013; 89(1):7.
82 Taylor HS. Endometrial cells derived from donor stem cells in bone marrow transplant recipients. JAMA. 2004; 292(1):81-5.
83 Schachinger V, Erbs S, Elsasser A, Haberbosch W, Hambrecht R, Holschermann H, et al. Intracoronary bone marrow-derived progenitor cells in acute myocardial infarction. N Engl J Med. 2006; 355(12):1210-21.
84 Brenner C, Adrion C, Grabmaier U, Theisen D, von Ziegler F, Leber A, et al. Sitagliptin plus granulocyte colony-stimulating factor in patients suffering from acute myocardial infarction: A double-blind, randomized placebo-controlled trial of efficacy and safety (SITAGRAMI trial). Int J Cardiol. 2016; 205:23-30.
83 Santamaria X, Cabanillas S, Cervello I, Arbona C, Raga F, Ferro J, et al. Autologous cell therapy with CD133+ bone marrow-derived stem cells for refractory Asherman's syndrome and endometrial atrophy: a pilot cohort study. Hum Reprod. 2016; 31(5):1087-96.
84 Alba M, Sheng D, Guan Y, Williams-Herman D, Larson P, Sachs JR, et al. Sitagliptin 100 mg daily effect on DPP-4 inhibition and compound-specific glycemic improvement. Curr Med Res Opin. 2009; 25(10):2507-14.
85 Farag SS, Nelson R, Cairo MS, O′Leary HA, Zhang S, Huntley C, et al. High-dose sitagliptin for systemic inhibition of dipeptidylpeptidase-4 to enhance engraftment of single cord umbilical cord blood transplantation. Oncotarget. 2017; 8(66):110350-7.

### EMBODIMENTS

1. A method for assessing the risk of pregnancy loss or embryo implantation failure in an individual, wherein the method comprises detecting and/or quantifying the amount of at least one marker gene for decidual cells and at least one marker gene for decidual senescent cells in a biological sample obtained from the individual, and thereby assessing the risk, wherein the at least one marker gene for decidual cells comprises Phospholipase A2 Group IIA (PLA2G2A).
2. The method according to embodiment 1, wherein the at least one marker gene for decidual cells further comprises Scavenger Receptor Class A Member 5 (SCARA5), Ferritin Light Chain (FTL), Glutaredoxin (GLRX) and/or Interleukin 1 Receptor Like 1 (IL1RL1)
3. The method according to any one of the preceding embodiments, wherein the at least one marker gene for decidual senescent cells is selected from Iodothyronine Deiodinase 2 (DIO2), Clusterin (CLU) and Insulin Like Growth Factor Binding Protein 1 (IGFBP1).
4. The method according to embodiment 3, wherein the at least one marker gene for decidual senescent cells is DIO2.
5. The method according to any one of the preceding embodiments, wherein the method comprises detecting and/or quantifying the amount of PLA2G2A and DIO2.
6. The method according to any one of the preceding embodiments, wherein a decreasing level of the marker gene for decidual cells, as compared with a reference sample or level, and/or
an increasing level of the marker gene for decidual senescent cells, as compared with a reference sample or level,
indicates that the individual is at risk of pregnancy loss or embryo implantation failure.

7. The method according to any one of the preceding embodiments, further comprising detecting and/or quantifying the level of uterine natural killer (uNK) cells or the level of at least one marker gene for uNK cells in the biological sample.

8. The method according to embodiment 7, wherein a decreasing level of uNK cells or level of the at least one marker gene for uNK cells, as compared with a reference sample or level, indicates that the individual is at risk of pregnancy loss or embryo implantation failure.
9. The method according to any one of the preceding embodiments, wherein the method further comprises a step of determining one or more risk indicia selected from the group consisting of maternal body mass index, maternal age and number of previous pregnancy losses or embryo implantation failures.

10. A method of monitoring or evaluating the effect of a treatment to reduce the risk of pregnancy loss or embryo implantation failure in an individual, wherein the method comprises detecting and/or quantifying the amount of at least one marker gene for decidual cells and at least one marker gene for decidual senescent cells in a biological sample obtained from the individual, and thereby monitoring or evaluating the effect of the treatment, wherein the at least one marker gene for decidual cells comprises PLA2G2A.
11. The method according to embodiment 10, wherein the at least one marker gene for decidual cells further comprises SCARA5, FTL, GLRX and/or IL1RL1.
12. The method according to embodiments 10 or 11, wherein the at least one marker gene for decidual senescent cells is selected from DIO2, CLU and IGFBP1.
13. The method according to embodiment 12, wherein the at least one marker gene for decidual senescent cells is DIO2.
14. The method according to any one of embodiments 10 to 13, wherein the method comprises detecting and/or quantifying the amount of PLA2G2A and DIO2.
15. The method according to any one of embodiments 10 to 14, wherein
(i) an increased level of the marker gene for decidual cells, as compared with a reference sample or level, and/or a decreased level of the marker gene for decidual senescent cells, as compared with a reference sample or level, indicates a positive response to treatment; and
(ii) a decreased level of the marker gene for decidual cells, as compared with a reference sample or level, and/or an increased level of the marker gene for decidual senescent cells, as compared with a reference sample or level, indicates a negative response to treatment.
16. The method according to any one of embodiments 10 to 15, further comprising detecting and/or quantifying the level of uterine natural killer (uNK) cells or the level of at least one marker gene for uNK cells in the biological sample.
17. The method according to embodiment 16, wherein (i) an increased level of uNK cells or the at least one marker gene for uNK cells, as compared with a reference sample or level, indicates a positive response to treatment; and (ii) a decreased level of uNK cells or the at least one marker gene for uNK cells, as compared with reference sample or level, indicates a negative response to treatment.
18. The method according to any one of embodiments 10 to 17, wherein the method comprises comparing the level of the marker genes at a first time point before or during the treatment, with the level of the marker genes at a later time point during or after the treatment.
19. The method according to any one of the preceding embodiments, wherein the risk of pregnancy loss is risk of euploid pregnancy loss, or wherein the risk of embryo implantation failure is not due to chromosomal abnormalities in an embryo.
20. The method according to any one of the preceding embodiments, wherein the risk of pregnancy loss is risk of recurrent pregnancy loss.
21. A method of diagnosing a reproductive disorder in an individual, wherein the method comprises detecting and/or quantifying the amount of at least one marker gene for decidual cells and at least one marker gene for decidual senescent cells in a biological sample obtained from the individual, thereby diagnosing the disorder, wherein the at least one marker for decidual cells comprises PLA2G2A.
22. The method according to embodiment 21, wherein the reproductive disorder is embryo implantation failure, pregnancy loss, recurrent miscarriage, recurrent pregnancy loss, or a placental disorder.
23 The method according to embodiment 21 or embodiment 22, wherein the at least one marker gene for decidual cells further comprises SCARA5, FTL, GLRX and/or IL1RL1.
24. The method according to any one of embodiments 21 to 23, wherein the at least one marker gene for decidual senescent cells is selected from DIO2, CLU and IGFBP1.
25. The method according to embodiment 24, wherein the at least one marker gene for decidual senescent cells is DIO2.
26. The method according to any one of embodiments 21 to 25, wherein the method comprises detecting and/or quantifying the amount of PLA2G2A and DIO2.
27. The method according to any one of embodiments 21 to 26, wherein
a decreased level of the decidual cell marker genes, as compared with a reference sample or level, and/or
an increased level of the decidual senescent cell marker genes, as compared with a reference sample or level,
indicates a positive diagnosis.
28. The method according to any one of embodiments 21 to 27, further comprising detecting and/or quantifying the level of uterine natural killer (uNK) cells or the level of at least one marker gene for uNK cells in the biological sample.
29 The method according to embodiment 28, wherein a decreased level of uNK cells or the at least one marker gene for uNK cells, as compared with a reference sample or level, indicates a positive diagnosis.
30. The method according to any one of embodiments 27 to 29, wherein in a positive diagnosis the ratio of PLA2G2A to DIO2 is:
(i) below the 50^{th} percentile as compared with a reference sample or reference level; or
(ii) below the 30^{th} percentile as compared with a reference sample or reference level.
31. The method according to any one of the preceding embodiments, wherein the biological sample is an endometrial biopsy sample.
32. The method according to any one of the preceding embodiments, wherein the biological sample is taken during the luteal phase of the menstrual cycle, optionally wherein the biological sample is taken during the mid-luteal phase of the menstrual cycle.
33. The method according to any one of the preceding embodiments, wherein the individual suffers or has suffered from infertility or embryo implantation failure following in vitro fertilisation treatment.
34. The method according to any one of the preceding embodiments, wherein the individual has already suffered from at least one previous pregnancy loss or embryo implantation failure, or is suffering from recurrent pregnancy loss.
35. The method according to any one of the preceding embodiments, wherein the method further comprises detecting and/or quantifying genes that allow identification of the day in the menstrual cycle, optionally wherein the genes that allow identification of the timing of the day in the menstrual cycle comprise, consist of, or consist essentially of, Glutathione Peroxidase 3 (GPX3) and Solute Carrier Family 15 Member 2 (SLC15A2).
36. The method according to any one of the preceding embodiments, wherein the marker genes are detected and/or quantified using ELISA, Western blotting, immunohistochemistry, immunoassays, enzymatic assays or sequencing methods, optionally wherein the sequencing methods include qPCR, Taqman-PCR, multiplex Taqman-PCR, Nanostring, targeted sequencing or digital PCR.
37. The method according to embodiment 36, wherein the digital PCR is digital droplet PCR (ddPCR).
38. A method of treating a reproductive disorder in an individual, the method comprising diagnosing the reproductive disorder according to the method of any one of embodiments 21 to 37, and administering an agent or carrying out a treatment regimen effective to treat the reproductive disorder in the individual who is positively diagnosed.
39. The method according to embodiment 38, wherein the agent or treatment regimen increases the level of decidual cells and/or uNK cells, and/or decreases the level of decidual senescent cells in the individual.
40. The method according to embodiment 38 or 39, wherein the agent is a DPP4 inhibitor.
41. The method according to embodiment 40, wherein the DPP4 inhibitor is sitagliptin.
42. The method according to any one of embodiments 38 to 41, wherein the individual has an increased level of at least one marker gene for decidual senescent cells.
43. The method according to any one of embodiments 38 to 42, wherein the at least one marker gene for decidual senescent cells is DIO2.
44. The method according to any one of embodiments 38 to 43, comprising administering progesterone and/or progestogen.
45. The method of any one of embodiments 21-44, wherein the reproductive disorder is recurrent pregnancy loss.
46. A method of selecting patients for treatment to reduce risk of embryo implantation failure or pregnancy loss, wherein the method comprises detecting and/or quantifying the amount of at least one marker gene for decidual cells and at least one marker gene for decidual senescent cells in a biological sample obtained from an individual, and selecting the patients for treatment to reduce the risk of pregnancy loss or embryo implantation failure based on the level of the marker genes, wherein the at least one marker for decidual cells comprises PLA2G2A.
47. The method according to embodiment 46, wherein an individual in which an increased level of at least one marker gene for decidual senescent cells is detected is selected for treatment.
48. The method according to embodiment 47, wherein the patient is selected for treatment with a DPP4 inhibitor
49. The method according to embodiment 48, wherein the DPP4 inhibitor is sitagliptin.
50. The method according to any one of embodiments 46 to 49, wherein the at least one marker gene for decidual senescent cells is DIO2.
51. The method according to embodiments 46 to 50, wherein the method of selecting patients for treatment is to reduce the risk of pregnancy loss, and wherein the pregnancy loss is recurrent pregnancy loss.
52. A test kit suitable for use in a method of any one of the preceding embodiments, wherein the test kit comprises means for detecting or quantifying at least one marker gene for decidual cells and at least one marker gene for decidual senescent cells at a nucleic acid or protein level, and optionally means for detecting and/or quantifying the level of UNK cells or the level of at least one marker gene for uNK cells in the individual, wherein the at least one marker gene for decidual cells comprises PLA2G2A.

1. A method for assessing the risk of pregnancy loss or embryo implantation failure in an individual, wherein the method comprises detecting and/or quantifying the amount of at least one marker gene for decidual cells and at least one marker gene for decidual senescent cells in a biological sample obtained from the individual, and thereby assessing the risk, wherein the at least one marker gene for decidual cells comprises Phospholipase A2 Group IIA (PLA2G2A).

2. The method according to claim 1, wherein
(a) the at least one marker gene for decidual cells further comprises Scavenger Receptor Class A Member 5 (SCARA5), Ferritin Light Chain (FTL), Glutaredoxin (GLRX) and/or Interleukin 1 Receptor Like 1 (IL1RL1); and/or
(b) the at least one marker gene for decidual senescent cells is selected from Iodothyronine Deiodinase 2 (DIO2), Clusterin (CLU) and Insulin Like Growth Factor Binding Protein 1 (IGFBP1), optionally wherein the at least one marker gene for decidual senescent cells is DIO2; and/or
(c) the method comprises detecting and/or quantifying the amount of PLA2G2A and DIO2.

3. The method according to any one of the preceding claims, wherein a decreasing level of the marker gene for decidual cells, as compared with a reference sample or level, and/or
an increasing level of the marker gene for decidual senescent cells, as compared with a reference sample or level,
indicates that the individual is at risk of pregnancy loss or embryo implantation failure.

4. The method according to any one of the preceding claims, further comprising
(a) detecting and/or quantifying the level of uterine natural killer (uNK) cells or the level of at least one marker gene for uNK cells in the biological sample, optionally wherein a decreasing level of uNK cells or level of the at least one marker gene for uNK cells, as compared with a reference sample or level, indicates that the individual is at risk of pregnancy loss or embryo implantation failure; and/or
(b) a step of determining one or more risk indicia selected from the group consisting of maternal body mass index, maternal age and number of previous pregnancy losses or embryo implantation failures.

5. A method of monitoring or evaluating the effect of a treatment to reduce the risk of pregnancy loss or embryo implantation failure in an individual, wherein the method comprises detecting and/or quantifying the amount of at least one marker gene for decidual cells and at least one marker gene for decidual senescent cells in a biological sample obtained from the individual, and thereby monitoring or evaluating the effect of the treatment, wherein the at least one marker gene for decidual cells comprises PLA2G2A.

6. The method according to claim 5, wherein
(a) the at least one marker gene for decidual cells further comprises SCARA5, FTL, GLRX and/or IL1RL1; and/or
(b) the at least one marker gene for decidual senescent cells is selected from DIO2, CLU and IGFBP1, optionally wherein the at least one marker gene for decidual senescent cells is DIO2; and/or
(c) the method comprises detecting and/or quantifying the amount of PLA2G2A and DIO2.

7. The method according to claim 5 or 6, wherein
(i) an increased level of the marker gene for decidual cells, as compared with a reference sample or level, and/or a decreased level of the marker gene for decidual senescent cells, as compared with a reference sample or level, indicates a positive response to treatment; and
(ii) a decreased level of the marker gene for decidual cells, as compared with a reference sample or level, and/or an increased level of the marker gene for decidual senescent cells, as compared with a reference sample or level, indicates a negative response to treatment.

8. The method according to any one of claims 5 to 7, wherein
(a) the method further comprises detecting and/or quantifying the level of uterine natural killer (uNK) cells or the level of at least one marker gene for uNK cells in the biological sample, optionally wherein (i) an increased level of uNK cells or the at least one marker gene for uNK cells, as compared with a reference sample or level, indicates a positive response to treatment; and (ii) a decreased level of uNK cells or the at least one marker gene for uNK cells, as compared with reference sample or level, indicates a negative response to treatment; and/or
(b) the method comprises comparing the level of the marker genes at a first time point before or during the treatment, with the level of the marker genes at a later time point during or after the treatment.

9. The method according to any one of the preceding claims, wherein
(a) the risk of pregnancy loss is risk of euploid pregnancy loss, or wherein the risk of embryo implantation failure is not due to chromosomal abnormalities in an embryo; and/or
(b) the risk of pregnancy loss is risk of recurrent pregnancy loss.

10. A method of diagnosing a reproductive disorder in an individual, wherein the method comprises detecting and/or quantifying the amount of at least one marker gene for decidual cells and at least one marker gene for decidual senescent cells in a biological sample obtained from the individual, thereby diagnosing the disorder, wherein the at least one marker for decidual cells comprises PLA2G2A.

11. The method according to claim 10, wherein
(a) the reproductive disorder is embryo implantation failure, pregnancy loss, recurrent miscarriage, recurrent pregnancy loss, or a placental disorder; and/or
(b) the at least one marker gene for decidual cells further comprises SCARA5, FTL, GLRX and/or IL1RL1; and/or
(c) the at least one marker gene for decidual senescent cells is selected from DIO2, CLU and IGFBP1, optionally wherein the at least one marker gene for decidual senescent cells is DIO2; and/or
(d) the method comprises detecting and/or quantifying the amount of PLA2G2A and DIO2.

12. The method according to claim 10 or 11, wherein
a decreased level of the decidual cell marker genes, as compared with a reference sample or level, and/or
an increased level of the decidual senescent cell marker genes, as compared with a reference sample or level,
indicates a positive diagnosis.

13. The method according to any one of claims 10 to 12, further comprising detecting and/or quantifying the level of uterine natural killer (uNK) cells or the level of at least one marker gene for uNK cells in the biological sample, optionally wherein a decreased level of uNK cells or the at least one marker gene for uNK cells, as compared with a reference sample or level, indicates a positive diagnosis.

14. The method according to claim 12 or 13, wherein in a positive diagnosis the ratio of PLA2G2A to DIO2 is:
(i) below the 50^{th} percentile as compared with a reference sample or reference level; or
(ii) below the 30^{th} percentile as compared with a reference sample or reference level.

15. The method according to any one of the preceding claims, wherein
(a) the biological sample is an endometrial biopsy sample; and/or
(b) the biological sample is taken during the luteal phase of the menstrual cycle, optionally wherein the biological sample is taken during the mid-luteal phase of the menstrual cycle.

16. The method according to any one of the preceding claims, wherein the individual
(a) suffers or has suffered from infertility or embryo implantation failure following in vitro fertilisation treatment; and/or
(b) has already suffered from at least one previous pregnancy loss or embryo implantation failure, or is suffering from recurrent pregnancy loss.

17. The method according to any one of the preceding claims, wherein
(a) the method further comprises detecting and/or quantifying genes that allow identification of the day in the menstrual cycle, optionally wherein the genes that allow identification of the timing of the day in the menstrual cycle comprise, consist of, or consist essentially of, Glutathione Peroxidase 3 (GPX3) and Solute Carrier Family 15 Member 2 (SLC15A2); and/or
(b) the marker genes are detected and/or quantified using ELISA, Western blotting, immunohistochemistry, immunoassays, enzymatic assays or sequencing methods, optionally wherein the sequencing methods include qPCR, Taqman-PCR, multiplex Taqman-PCR, Nanostring, targeted sequencing or digital PCR, optionally wherein the digital PCR is digital droplet PCR (ddPCR).

18. A method of treating a reproductive disorder in an individual, the method comprising diagnosing the reproductive disorder according to the method of any one of claims 10 to 17, and administering an agent or carrying out a treatment regimen effective to treat the reproductive disorder in the individual who is positively diagnosed.

19. The method according to claim 18, wherein
(a) the agent or treatment regimen increases the level of decidual cells and/or uNK cells, and/or decreases the level of decidual senescent cells in the individual.
(b) the agent is a DPP4 inhibitor, optionally wherein the DPP4 inhibitor is sitagliptin; and/or
(c) the individual has an increased level of at least one marker gene for decidual senescent cells; and/or
(d) the at least one marker gene for decidual senescent cells is DIO2; and/or
(e) the method comprises administering progesterone and/or progestogen.

20. The method of any one of claims 10-19, wherein the reproductive disorder is recurrent pregnancy loss.

21. A method of selecting patients for treatment to reduce risk of embryo implantation failure or pregnancy loss, wherein the method comprises detecting and/or quantifying the amount of at least one marker gene for decidual cells and at least one marker gene for decidual senescent cells in a biological sample obtained from an individual, and selecting the patients for treatment to reduce the risk of pregnancy loss or embryo implantation failure based on the level of the marker genes, wherein the at least one marker for decidual cells comprises PLA2G2A

22. The method according to claim 21, wherein an individual in which an increased level of at least one marker gene for decidual senescent cells is detected is selected for treatment, optionally wherein the patient is selected for treatment with a DPP4 inhibitor, further optionally wherein the DPP4 inhibitor is sitagliptin.

23. The method according to claim 21 or 22, wherein
(a) the at least one marker gene for decidual senescent cells is DIO2; and/or
(b) the method of selecting patients for treatment is to reduce the risk of pregnancy loss, and wherein the pregnancy loss is recurrent pregnancy loss.

24. A test kit suitable for use in a method of any one of the preceding claims, wherein the test kit comprises means for detecting or quantifying at least one marker gene for decidual cells and at least one marker gene for decidual senescent cells at a nucleic acid or protein level, and optionally means for detecting and/or quantifying the level of UNK cells or the level of at least one marker gene for uNK cells in the individual, wherein the at least one marker gene for decidual cells comprises PLA2G2A.
